(19) United States
(12) Patent Application Publication
(10) Pub. No.: US 2017/0239306 A1
(43) Pub. Date: Aug. 24, 2017

(54) SOYBEAN EXTRACTS AND COMBINATIONS THEREOF WITH POLYETHOXYLATED CASTOR OIL AND OTHER ADJUVANTS FOR CONTROLING BLOOD SUGAR LEVELS AND FOR HEPATOPROTECTION

(71) Applicant: NATURAL SHIELD ISRAEL 2016 LTD, Kfar Tavor (IL)

(72) Inventor: Yaron ILAN, Kfar Tavor (IL)

(73) Assignee: NATURAL SHIELD ISRAEL 2016 LTD, Kfar Tavor (IL)

(21) Appl. No.: 15/506,279

(22) PCT Filed: Aug. 11, 2015
(86) PCT No.: PCT/IL2015/050821

§ 371 Date: Feb. 24, 2017

Related U.S. Application Data
(60) Provisional application No. 62/041,201, filed on Aug. 25, 2014.

(51) Int. Cl. A61K 36/48; A61K 47/10; A61K 47/40; A23L 33/115; A61K 31/685; A61K 31/661; A23L 2/52; A23L 33/105; A61K 47/44; A61K 45/06

(52) U.S. Cl. CPC A61K 36/48; A61K 47/44; A61K 47/10; A61K 47/40; A61K 45/06; A61K 31/685; A61K 31/661; A23L 2/52; A23L 33/105; A23L 33/115; A23V 2002/00

(57) ABSTRACT

Provided are compositions and methods using soy extracts and fractions thereof, optionally in combination with castor oil, for controlling blood sugar levels in a subject, treating liver damage and restoring liver function, treating, preventing, ameliorating, reducing or delaying the onset of acute or chronic toxic effect of a drug, treating an immune related disorder, and for enhancing the therapeutic effect of a therapeutic agent in a subject.

### FIELD OF INVENTION

The present invention relates to food supplements and therapeutic compositions for controlling blood sugar levels, protecting and restoring liver function. More particularly, the invention relates to combined therapeutic compositions and food supplements comprising soy extracts and polyethoxylated castor oils and optionally other adjutants such as polyethylene glycol or beta cyclo dextrin.

### BACKGROUND REFERENCES

1. Gallup's Annual Consumption Habits Poll, Jul. 9-12, 2012 http://www.gallup.com/Search/Default.aspx?q=consumption+habits+poll+July+9-12
2. American Heart Association Facts Sheets http://www.heart.org/idc/groups/heart-public/@wcm/@adv/documents/downloadable/ucm_453908.pdf
3. Ludwig D S, et al. Relation between consumption of sugar-sweetened drinks and childhood obesity: a prospective, observational analysis. The Lancet, Volume 357, Issue 9255, pages 505-508 (2001).
4. Schulze M B, et al. Sugar-sweetened beverages, weight gain, and incidence of type 2 diabetes in young and middle-aged women. JAMA, Volume 292, Issue 8, pages 927-934 (2004).
5. Dhingra R et al. Soft drink consumption and risk of developing cardiometabolic risk factors and the metabolic syndrome in middle-aged adults in the community. Circulation, Volume 116, Issue 5, pages 480-488 (2007).
6. Global Burden of Disease 2010 http://rt.com/usa/soda-sugar-danger-research-singh-559
7. CDC Facts Sheets http://www.cdc.gov/alcohol/fact-sheets/alcohol-use.htm
8. Swift R and Davidson D. Alcohol hangover: mechanisms and mediators. Alcohol Health & Research World, Volume 22, Issue 1, pages 54-60 (1998)
9. Menon K V et al. Pathogenesis, diagnosis, and treatment of alcoholic liver disease. Mayo Clinic Proceedings, Volume 76, Issue 10, pages 1021-1029 (2001).
10. O'Shea R S et al. Alcoholic liver disease: AASLD Practice Guidelines. Hepatology, Volume 51, Issue 1, pages 307-328 (2010).
11. Morgan M Y. The prognosis and outcome of alcoholic liver disease. Alcohol and Alcoholism, Volume 2 (Suppl), pages 335-343 (1994).
12. Erickson S K. Nonalcoholic fatty liver disease. J Lipid Res, Volume 50 (Suppl), pages S412-S416 (2009).

### BACKGROUND OF THE INVENTION

Stability of the level of blood glucose (or blood sugar) is the basic prerequisite for maintenance of controlled influx and availability of glucose to the cells. Glucose, being the preferential source of energy in virtually all body cells, is essential for normal function of all body systems, which is why blood glucose levels are tightly regulated as a part of metabolic homeostasis governed by pancreatically produced insulin/glucagon feedback. In all vertebrates, regardless of large fluctuations in physical activity and food intake, blood sugar levels are held within very narrow limits. In humans, the normal blood glucose levels (tested while fasting) for non-diabetics, are on average between 70-100 milligrams per deciliter (mg/dL). Blood glucose levels outside the normal range, i.e. persistent hyper- or hypo-glycemia, may be an indicator of a number of medical conditions. Diabetes mellitus characterized by persistent hyperglycemia and is the most prominent disease related to failure of blood sugar regulation. Intake of alcohol causes an initial surge in blood sugar, and later tends to cause hypoglycemia.

Apart from issues of lifestyle and self esteem, controlling blood sugar levels and maintaining a healthy weight are vital to lower the risk of diseases such as type 2 diabetes (the most common adult form of diabetes resulting from insulin resistance), morbid obesity, heart disease, liver disease and cancer. Consequences of chronic alcohol consumption are numerous, apart from risks of injuries sustained in car accidents and liver cirrhosis, there are also risks of anemia, cardiovascular disease, cancer and distinct neurologic and psychiatric disorders. Sugar enriched foods, particularly soft drinks, and alcohol are considered among major health hazards produced by a modern way of living. The Gallup's Annual Consumption Habits Poll conducted in Jul. 9-12, 2012 in the US, for example, indicated that about half of all Americans, 48%, consume on average at least one glass of a soda per day and 66%—over four alcoholic drinks per week [1].

According to the American Heart Association, soft drinks and other sugar-sweetened beverages (SSBs) are the primary source of added sugars in Americans' diets; their increased consumption has been associated with rising obesity rates. Consumption of SSBs has increased 500% in the past fifty years and is now the single largest category of caloric intake in children, about 10-15% of the total daily calorie intake [2]. The rising prevalence of obesity in children has been linked, in part, to the consumption of SSBs [3]. Consumption of excessive calories and large amounts of rapidly absorbable sugars through SSBs was recognized as one of significant contributors to weight gain and incidence of type 2 diabetes in American women between 1991 to 1999 [4]. In fact, individuals consuming one or more SSB per day have higher odds for developing metabolic syndrome (odds ratio OR=1.48), obesity (OR=1.31), increased waist circumference (OR=1.30), impaired fasting glucose (OR=1.25), higher blood pressure (OR=1.18), hyper-triglyceridemia (OR=1.25), and low high-density lipoprotein cholesterol (OR=1.32) [5]. A recent research by the Harvard School of Public Health summarizing data for Global Burden of Disease for 2010, suggested that SSBs were directly responsible for 133,000 deaths from diabetes, 44,000 deaths from cardiovascular disease and 6,000 deaths from cancer worldwide and for a total of 25,000 deaths in the US alone [6].

Concerns with regard to excessive sugar and alcohol consumption imposed by the modern life style are clear. Public health policy makers and professionals are currently conducting a number of policies to control consumption, including taxation and legislation. The food and beverage industry is increasingly replacing sugary products with sugar-free or artificially sweetened versions. There is however apparent shortage of candidate food additives, natural or synthetic, having potential to counter-balance the negative effects of both, excess sugar and alcohol. Two food additives getting lately a lot of attention as blood sugar busting components are vinegar and cinnamon.

According to the Centers for Disease Control (CDC) statistics for 2006-2010, there are annually 88,000 deaths attributable to excessive alcohol consumption in the US alone, making alcohol the 3rd leading lifestyle-related cause of death in the nation. In US 2006, for example, there were more than 1.2 million emergency room visits and 2.7 million physician office visits due to excessive drinking [7]. As previously mentioned, excessive alcohol consumption has immediate effects on many harmful health conditions, such as in increasing risk of injuries, violence (about 35% of violence victims report that offenders were under the influence of alcohol), risky sexual behavior and unprotected sex, miscarriage and stillbirth among pregnant women. Over time, excessive alcohol use can lead to the development of chronic diseases, including liver disease, alcoholic hepatitis and cirrhosis, the latter are among the leading causes of deaths in the US. Long-term health risks also include, but are not limited to, neurological impairments, cardiovascular problems, and psychiatric and social problems.

Several alterations in the metabolic state of the liver and other organs occur in response to the presence of alcohol (ethanol) in the body and can result in low blood sugar levels (hypoglycemia) [8]. Alcohol metabolism leads to a fatty liver and buildup of an intermediate metabolic product, lactic acid, in body fluids (lactic acidosis). Both of these effects can inhibit glucose production. Alcohol-induced hypoglycemia generally occurs after prolonged alcohol consumption coupled with poor nutritional intake, which not only decreases glucose production but also exhaust the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Excessive alcohol consumption is the major cause of liver disease; 15-20% of chronic heavy drinkers develop hepatitis or cirrhosis that can occur concomitantly or in succession. While genetic factors may contribute both to alcoholism and to alcoholic liver disease, malnutrition, particularly vitamin A and E deficiencies, can worsen alcohol-induced liver damage by preventing hepatocyte regeneration [9]. Women are twice as susceptible to alcohol-related liver disease, and may develop alcoholic liver disease with shorter durations and doses of chronic consumption. Alcoholic liver disease evolves as a result of secretion of pro-inflammatory cytokines, oxidative stress, lipid peroxidation and acetaldehyde toxicity ensuing in response to alcohol consumption. These factors cause inflammation, apoptosis and eventually fibrosis of liver cells [10].

Alcoholic liver disease evolves from fatty change through alcoholic hepatitis to alcoholic cirrhosis. Its development is associated with an excess mortality both in relation to the presence of liver disease and to other complications of alcohol abuse. In the majority of patients fatty liver is a benign lesion, which will reverse completely following abstinence from alcohol. Continued drinking is associated with the eventual development of cirrhosis in approximately 20% of individuals. Alcoholic hepatitis is a precirrhotic lesion; progression to cirrhosis is observed more commonly in women, in individuals with severe disease and in those who continue to drink. Thirty-day mortality rates of less than 20% are observed in patients with mild to moderate disease but exceed 40% in individuals with severe liver injury. Survival is significantly reduced in women and in the elderly and is adversely affected by the presence of severe liver injury, evolution to cirrhosis and continued drinking Two-thirds of patients with alcoholic cirrhosis present with decompensated disease; 15% will develop hepatocellular carcinoma. Survival is adversely affected by the presence of decompensated disease, superimposed alcoholic hepatitis, continued drinking and the development of hepatocellular carcinoma [11].

Nonalcoholic fatty liver disease (NAFLD) is rapidly becoming a worldwide public health problem. It is the most common liver disease in the US and, indeed, worldwide. Current estimates are that ˜20% of the general US population has NAFLD. The prevalence in the morbidly obese population has been estimated as 75-92%, while that in the pediatric population as 13-14%. At present, it is estimated that ˜6 million individuals in the US general population have progressed to nonalcoholic steatohepatitis (NASH) and ˜600,000 to NAFLD-related cirrhosis. Thus, the number of individuals at risk for end-stage liver disease and development of primary liver cancer and those potentially eligible for liver transplant is large. Prevalence of NAFLD appears to be increasing, in part due to the increasing numbers of adult and pediatric individuals who are either obese or overweight, have metabolic syndrome or type 2 diabetes, all major risk factors for development of NAFLD [12].

WO 2012/017435 is a previous publication of the present inventor that describes methods and uses of different soybean extracts for the treatment of hepatic disorders, drug induced hepatic injury and related metabolic disorders.

Thus, there is a major need for therapeutic compounds, food supplements, food additives, medical foods, botanical drugs and safe drugs assisting in control of blood sugar levels and thereby facilitating prevention and amelioration of related disorders.

### SUMMARY OF INVENTION

A first aspect of the invention relates to compositions for least one of, controlling blood sugar levels in a subject, treating an immune related disorder, treating liver damage and restoring liver function, and treating, preventing, ameliorating, reducing or delaying the onset of acute or chronic toxic effect of a drug, and enhancing the therapeutic effect of a therapeutic agent. More specifically, the compositions of the invention may comprise as an active ingredient at least one of:

(a) at least one soy extract (SE) or any fraction thereof;

(b) at least one polyethoxylated castor oil and/or optionally at least one adjuvant selected from polyethylene glycol and beta cyclo dextrin or any derivative thereof;

(c) any combination of (a) and (b); and

(d) a composition comprising any one of (a), (b) and (c).

A further aspect of the invention relates to a method for controlling blood sugar levels in a subject, treating liver damage, restoring liver function for treating, preventing, ameliorating, reducing or delaying the onset of acute or chronic toxic effect of alcohol consumption or of a drug, treating an immune related disorder, and for enhancing the therapeutic effect of a therapeutic agent. The method of the invention comprises the step of providing to a subject at least one of: (a) at least one soy extract or any fraction thereof; (b) at least one polyethoxylated castor oil or any derivative thereof and/or optionally at least one adjuvant selected from polyethylene glycol and beta cyclo dextrin or any derivative thereof; (c) any combination of (a) and (b); and (d) a composition comprising any one of (a), (b) or (c).

Still further, the invention provides a soft or an alcoholic beverage comprising a soy extract or any fraction thereof and optionally further comprising a polyethoxylated castor oil or any derivative or a combination thereof.

In yet another aspect, the invention provides a combined composition comprising as an active ingredient at least one soy derived polar fraction and at least one polyethoxylated castor oil or any derivative thereof.

These and other aspects of the invention will become apparent by the hand of the following description.

### BRIEF DESCRIPTION OF FIGURES

FIG. 1. Soy-derived extract (M1) supplementation to SSB lowers blood sugar levels The figure shows mean blood glucose levels at 0, 15, 30 and 60 min time points after consumption of SSB alone or with supplementation of M1 dissolved in water (DDW) or in 30% Cremophor EL (C:E) or 30% C:E solution per se. Experimental conditions are detailed in Table 1.

FIG. 2. Long term effects of M1 supplementation to SSB on lowering blood sugar levels The figure shows mean blood glucose levels at 60, 90, 120 and 180 min time points after consumption of SSB alone or with supplementation of M1 dissolved in DDW or in 30% C:E or 30% C:E alone (with reference to experimental conditions detailed in Table 1).

FIG. 3. M1 supplementation to SSB improves glucose tolerance The figure shows a histogram representing Total Area Under the Curve (AUC) values of blood glucose levels from FIGS. 1 and 2 in various groups. Total AUC represents a pharmacokinetic estimate of the total glucose exposure overtime, which is particularly indicative of glucose tolerance.

FIG. 4. M1 and/or OS supplementation to SSB exerts long term control on blood glucose levels and improves glucose tolerance The figure shows a histogram representing AUC values of blood glucose levels at 120 and 180 min time points after consumption of SSB±M1 and/or OS supplementation (with reference to experimental conditions detailed in Table 2).

FIG. 5. Combination of M1, OS and/or C:E supplementation to SSB improves glucose tolerance The figure shows a histogram representing mean serum glucose levels at AUC values of blood glucose levels at 120 min time point after consumption of SSB±M1 and/or OS supplementation in DDW or 30% C:E (with reference to experimental conditions detailed in Table 3).

FIG. 6. Protective effects of M1 and C:E on immune-mediated hepatitis Figure shows a histogram representing serum Alanine transaminase (ALT) after treatment with M1±30% C:E in an animal model of liver damage induced by Concanavalin A (Con A) (with reference to experimental conditions in Table 4).

FIG. 7. Effect of oral co-administration of OS and M1 on the alcohol-mediated liver damage Figure shows AST levels at 16 hours in mice receiving orally ethanol (EtOH) or EtOH supplemented with OS M1. The results show a significant beneficial effect of oral co-administration of OS and M1 and alcohol in alleviating the alcohol-induced liver damage, suggesting that in these conditions OS and M1 act as liver protectors. (Group A are normal naïve mice, Group B: Received ethanol, Group C received ethanol with M1OS).

FIG. 8. Effect of oral co-administration of OS and M1 on the alcohol-mediated reduction in body weight Figure shows body weight in grams at 16 hours in mice receiving orally ethanol (EtOH) or EtOH supplemented with OS M1. The results show a significant beneficial effect of oral co-administration of OS and M1 and alcohol in alleviating the alcohol-induced reduction of bogy weight, suggesting that in these conditions OS and M1 act as protectors. (Group A are normal naïve mice, Group B: Received ethanol, Group C received ethanol with OS and M1).

FIG. 9A-9B. Effect of oral co-administration of OS and M1 on the alcohol-mediated alteration of regulatory T cells Figure shows percentages of NKT cells (CD3+NK1.1+, FIG. 9A) and CD4+CD25+Foxp3+ regulatory T cells (FIG. 9B) at 16 hours in mice receiving orally ethanol (EtOH) or EtOH supplemented with OS M1. The results show a significant beneficial effect of oral co-administration of OS and M1 and alcohol in reducing NKT cells which mediate the liver damage, and in correcting the redistribution of CD4+CD25+Foxp3+, suggesting that in these conditions OS and M1 act as immune balancers. (Group A is normal naïve mice, Group B: Received ethanol, Group C received ethanol with M1OS).

### DETAILED DESCRIPTION OF THE INVENTION

The present invention stems from findings by the inventors showing that various extracts of soy or soy derived fractions obtained by standard extraction methods possess surprising properties of lowering blood sugar/glucose levels and improving glucose tolerance, particularly when supplemented to food or beverages containing high sugar content (Examples 1 and 2). The inventors' findings further suggested that these properties could be enhanced by combining said soy derived extracts or fractions, with a synthetic castor oil derivative, i.e. polyethoxylated castor oil commercially known as Cremophor EL (C:E).

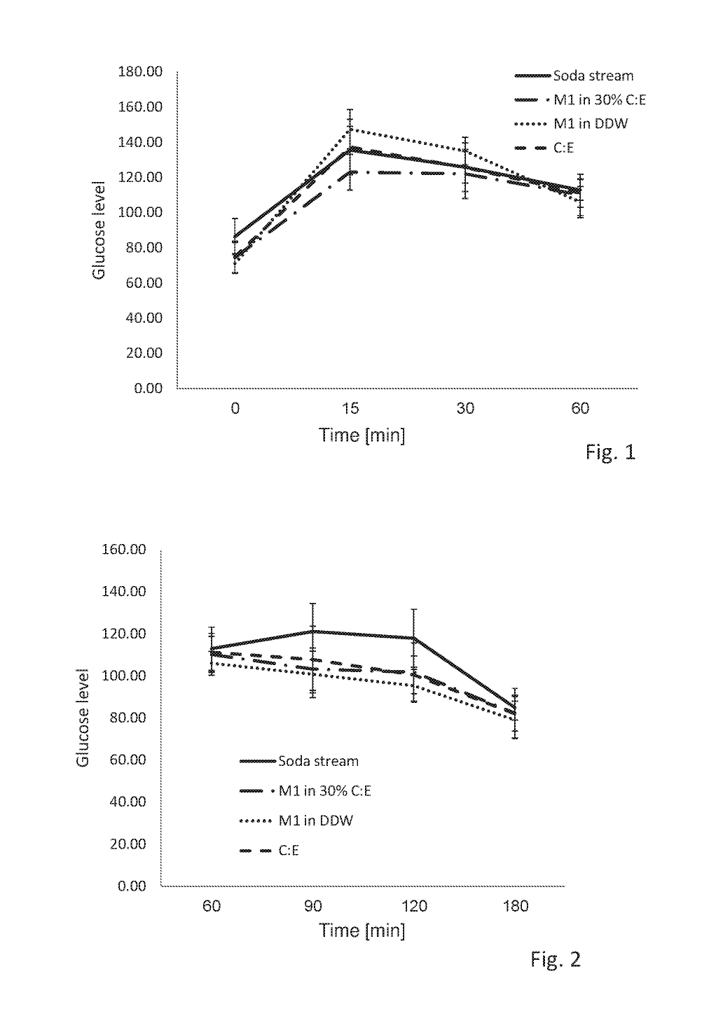

Specifically, the inventors have shown that supplementation of soy derived polar or non-polar fractions, designated M1 and OS respectively, alone or in combination with C:E, to beverages or foods with high sugar content prevents increase in blood/serum glucose levels and significantly reduces the total glucose exposure overtime and thus enables long term control on blood glucose levels and on glucose tolerance (FIGS. 1 to 5). Further, these findings being indicators of improved blood glucose clearance, which among others is governed by insulin-mediated glucose storage in the liver, also lead to the notion that soy derived polar or non-polar fractions, alone or in combination with C:E, may have beneficial effects on liver and/or pancreatic function.

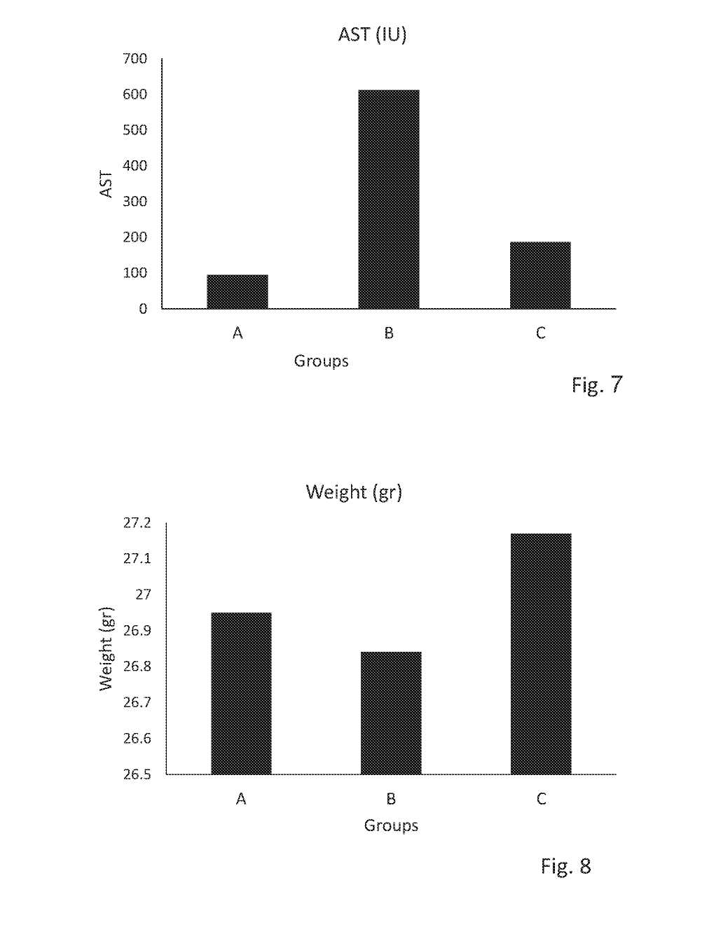

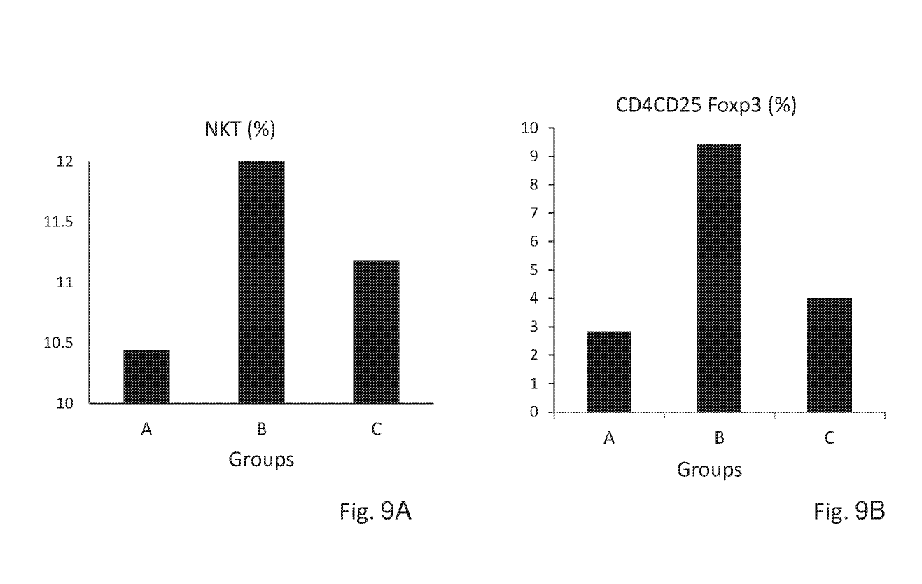

Indeed, in additional set of experiments, the inventors have demonstrated that one of the sites of action of a soy derived polar fraction (M1) is the liver (Example 3). Specifically, it has been presently demonstrated that M1 has protective effects on liver damage resulting from immune-mediated insult induced by Concanavalin A (Con A) (FIG. 6). Further, it has been demonstrated that a combination of M1 and C:E have an additive effect in restoring liver function and protecting from liver damage. Moreover, the protective effect of the composition of the invention has been demonstrated on alcohol induced liver damage model. More specifically, addition of the M1 and OS soy extracts, optionally with C:E, protected from reduction in body weight (FIG. 8), restored liver enzymes function (FIG. 7) and controlled alteration in regulatory T cells caused by alcohol consumption (FIG. 9).

These findings could be interpreted on several levels. One being that these new properties of compositions of the invention in preventing high blood glucose levels have direct therapeutic implications on a number of clinical conditions, particularly various types of diabetes, insulin resistance, disorders of carbohydrate metabolism and other related metabolic conditions. Further, the present invention may be perceived in a broader sense wherein compositions of the present invention serve basis for the development of new therapeutic compounds for treatment of hepatopathologies in a large number of clinical contexts, including alleviation of immuno-mediated liver damage, drug-induced liver damage, alcohol-induced liver damage, as well as cirrhosis and/or hepatic failure ensuing from infections, cancer, alcoholic steatohepatitis, non-alcoholic steatohepatitis (NASH or NAFLD) and other chronic liver diseases.

Not less important, these findings lead to notion that compositions of the present invention may be used as “bouncers” in preventing the development of pre-clinical conditions ensuing from exposure to exceeding increased or decreased blood sugar levels occurring after consumption of sugar-enriched foods and beverages or alcohol. In this context, compositions of the invention rather than being applicable to patients as therapeutic agents are implemented as food additives meant to normalize risks ensuing from modern lifestyle and standard of living to which are subjected normal individuals. Said therapeutic compounds, drugs, medical foods, food supplements and food additive, especially in form of add-on to sugar sweetened and/or alcoholic beverages are especially beneficial for preventing common conditions, such as weight gain, alcohol intoxication and risk of risk of cardiovascular pathology, and also more severe presentations, such as obesity and alcohol withdrawal syndrome.

By their nature, compositions using any soy derived extracts, including polar or non-polar fractions, as well as polyethoxylated castor oils or their derivatives, should be safe and lacking major adverse effects and thus may be applicable not only in a secondary prevention of already existing clinical disorders but also in a primary prevention of risks or pre-clinical conditions in a normal population.

Thus, it is conceived that in one of its aspects the present invention provides a composition for use in at least one of, a method for controlling blood sugar levels in a subject, a method of treating liver damage and restoring liver function, a method for the treatment of an immune related disorder, and a method for treating, preventing, ameliorating, reducing or delaying the onset of acute or chronic toxic effect of alcohol consumption or of a drug. More specifically, the composition of the invention comprises as an active ingredient at least one of:

(a) at least one soy extract (SE) or any fraction thereof;

(b) at least one polyethoxylated castor oil and/or optionally at least one adjuvant selected from polyethylene glycol and beta cyclo dextrin or any derivative thereof;

(c) any combination of (a) and (b); and

(d) a composition comprising any one of (a), (b) and (c).

For the purpose of specific applications of the invention according to the above, compositions of the invention may comprise as an active ingredient at least one soy extract (SE) or any fraction thereof and optionally comprise at least one polyethoxylated castor oil or any derivative thereof or any combination thereof, and/or optionally at least one adjuvant selected from polyethylene glycol and beta cyclo dextrin or any derivative thereof.

Under soy is meant any part of a plant belonging to the genus Glycine, including the two subgenera, Glycine and Soja. Seeds (also beans) or pollen of said plants are of particular applicability to the present invention. Further pertinent thereto, genetically modified soy, which may include, among others, glyphosate-tolerant or herbicide-tolerant soy that constitute now the majority of the commercial market (e.g. 93% in the US).

Further, the term extract refers to any substances obtained by extracting soy, particularly soybeans, using either enzymatic extracts, organic solvents or by hydrophilic solvents. More specifically, the term extract refers to any substances obtained by extracting soy using either organic solvents such as, for example, hexane, ethyl-acetate or isopropyl-alcohol, or by hydrophilic solvents, such as water. The extracts may be dried after said extraction and may be further extracts by any extraction method, independently from previous extraction steps. Such steps may be repeated independently. Furthermore, other extraction techniques may be employed, non-limiting examples of which include chromatography, including size-exclusion, hydrophobic interaction, and anion and cation exchangers, differential centrifugation, differential precipitation (for example, using ammonium sulfate), differential filtration and dialysis.

Many extraction methods may be used for producing SE of the invention. For example, at least one of an aliphatic organic solvent and water, or supercritical carbon dioxide gas may be used as an extractant for extraction of phospholipids from soybeans, preferably a defatted soybean material. The aliphatic organic solvent is preferably a saturated hydrocarbon, an alcohol, a mixed solvent of saturated hydrocarbon and alcohol, or a mixed solvent of halogenated hydrocarbon and alcohol. It is preferable that the extract be at least one of hexane, ethanol, methanol, hydrous ethanol, isopropyl alcohol, acetonitrile and acetone.

Further, SE may be enriched with aromatic chromophore containing compounds including the isoflavones genistein, daidzein, formononetin and biochanin and/or their glycosides, and for administration it is generally provided in association with one or more pharmaceutically acceptable carriers, excipients, auxiliaries, and/or diluents.

Other procedures for specifically enriching or removing soybean isoflavones include differential extraction with organic solvents, based on the differing solubility of aromatic chromophore containing compounds in certain organic solvents.

Apart from extracts derived from the soybean, other extracts may be derived from the solvent extraction of soy pollens into oil which contains tri- and di-glicerydes, free fatty acids and phosphatides, as well as extracts derived from aqueous-ethanol extraction left after the solvent extraction, which contains soy protein, isoflavones, sugars (oligo-, di-, mono-), and lipids (including phosphatides, phytosterols, saponins).

Thus, for the purpose of certain embodiments and methods of the invention, compositions of the invention may comprise any soy derived preparation extract.

As described in the art, SE may also incorporate enzymatic treatment of said soybeans, or other soy plant material, whether before, during or after mechanical disruption and/or chemical extraction of plants. Therefore, enzymatic treatment of the plant material is specifically contemplated herein. Enzymes used for said extraction include cellulase, hemicellulase, pectinase, protease and other carbohydrases. The use of enzymatic treatment may be carried out under various moisture and temperature conditions suitable for optimal enzyme activity as known in the art. When performing enzymatic treatment of the soy plant material during chemical extraction, it is appreciated that the solvent and conditions used must be compatible with the maintenance of adequate enzymatic activity, and care must be taken not to inhibit the enzyme activity or to denature it.

Of particular relevance to the present invention compositions comprising as an active ingredient a soy derived fraction which is either soy derived polar or non polar fraction. Said polar and/or non-polar fractions, may be in particular embodiments soy extract fractions presently designated as M1 and OS respectively. These specific fractions of SE may be obtained by standard processing procedures for extracting soy oil and soy protein. When subjected to qualitative LC-MS and ¹H-, ³¹P-NMR analyses, M1 and OS fractions can be identified with characteristic chemical profiles, as further detailed below.

For example, the M1 (polar) fraction can be obtained by standard hydro-alcoholic extraction of defatted soy milk to food soy protein. Specific constituents of M1 and OS fractions may be identified using qualitative LC-MS, ¹H-NMR analyses. For LC-MS analysis, the M1 fraction is dissolved in DMSO and analyzed on C-18 reversed column and polar mobile phase consisting of water (modified with ammonium formate) and methanol. For the ¹H-NMR analysis—the M1 fraction is dissolved in different solvents. According to both analyses, M1 is characterized by typical phosphatidylcholine (PC) and phosphatidylinositol (PI) content, in declining order. According to more accurate ³¹P-NMR analysis, M1 is characterized by a highly heterogeneous content of phospholipids and phosphatides. M1 is predominantly enriched in phosphatidylcholine (PC) and phosphatidylinositol (PI).

For LC/MS analysis, the OS (non-polar) fraction is dissolved in chloroform and analyzed on reversed column C-18 and non polar mobile phase consisting of methanol and ethyl acetate. According to LC/MS and NMR analyses, the OS fraction predominantly contains glycerides and phospholipids, in declining order. By ³¹P-NMR spectroscopy, OS is mainly enriched in phosphatidic acid (PA), phosphatidylethanolamine (PE) and phosphatidylcholine (PC). OS and M1 fractions are distinct by ratios of various phosphatides.

Knowing the specific constituents of M1 and OS fractions, it is conceived that certain compositions of the present invention may comprise not only natural but also synthetic M1 or OS fractions or any partial constituents thereof or any combination of said constituents.

Thus, in certain embodiments and methods according to the present invention, compositions of the present invention comprise any soy derived polar fraction comprising at least one of phospholipids, phosphatides or a combination thereof.

In further embodiments, said compositions comprising phosphatides, specifically comprise any one of phosphatidylcholine (PC), phosphatidylinositol (PI) or a combination thereof, which are characteristic of the polar fraction presently designated as M1.

More specific embodiments relate to a composition according to the invention comprising as an active ingredient the M1 soy extract.

It should be appreciated that in certain embodiments, the M1 extract may comprise the PC and PI and possibly any further compounds in any ratio, for example, 1:1 to 0.001:1000, specifically, 1:1, 1:2, 1:3, 1:4, 1:5, 1:6, 1:7, 1:8, 1:9, 1:10, 20, 30, 40, 50, 60, 70, 80, 90, 100, 200, 300, 400, 500, 600, 700, 800, 900, 1000 or more. In yet another alternative embodiment, the M1 extract may comprise each of the PI and PC in an amount ranging between 0.001% to 99.9%. More specifically, each of PI and/or PC may be present in the M1 extract of the invention in an amount of 0.001%, 0.005%, 0.01%, 0.05%, 0.1%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99% and more. It should be further appreciated that further ingredients may be present in said extracts as mentioned herein after including isoflavones, sugars and lipids.

In yet another particular embodiment, the composition of the invention may comprise as an active ingredient the M1 soy extract fraction and at least one polyethoxylated castor oil or any derivative thereof.

Yet in other embodiments and methods, compositions of the present invention comprise any soy derived non-polar fraction comprising at least one of glycerides, phospholipids and phosphatides.

In further embodiments, said compositions comprising at least one of glycerides, specifically comprise phospholipids and phosphatides are any one of phosphatidic acid (PA), phosphatidylethanolamine (PE) and phosphatidylcholine (PC), which are characteristic of the non-polar fraction presently designated as OS.

It should be appreciated that in certain embodiments, the OS extract may comprise the PE, PC and PA indicated above, and possibly any further compounds in any ratio, for example, 1:1:1 to 0.001:0.1:1000. In yet another alternative embodiment, the OS extract may comprise each of the PE, PC and PA in an amount ranging between 0.001% to 99.9%. More specifically, each of PE, PA and/or PC may be present in the OS extract of the invention in an amount of 0.001%, 0.005%, 0.01%, 0.05%, 0.1%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99% and more. It should be further appreciated that further ingredients may be present in said extracts as mentioned herein after including tri- and di-glycerides, free fatty acids and phosphatides.

More specific embodiments relate to a composition according to the invention comprising as an active ingredient the OS soy extract. Another specific embodiment relates to compositions comprising as an active ingredient a combination of the M1 and OS soy extract fractions.

In yet another particular embodiment, the composition of the invention may comprise as an active ingredient the OS soy extract fraction and at least one polyethoxylated castor oil or any derivative thereof. Still further specific embodiments of the invention relate to compositions comprising as an active ingredient a combination of the M1 and OS soy extract fractions and at least one polyethoxylated castor oil or any derivative thereof.

Another important active ingredient of compositions of the present invention, which is specifically applicable to certain embodiments and methods, is castor oil, more specifically a synthetic derivative thereof, i.e. a polyethoxylated castor oil or any derivative thereof.

Castor oil as meant herein relates to a natural vegetable oil obtained from seeds of the castor oil plant (Ricinus communis). The FDA has categorized castor oil as “generally recognized as safe and effective” (GRASE) for over-the-counter use as a laxative. Castor oil or synthetic castor oil derivatives such as polyethoxylated castor oil have been approved for human use as vehicles for oral and intravenous administration of water-insoluble therapeutic compounds. In naturopathy, castor oil has been promoted as a treatment for a variety of human health conditions.

The term ‘Ethoxylated Castor Oil’ (also Polyoxyl Castor Oil, Polyoxyl Castor Oil, Polyethylene Glycol Castor Oil, Castor Oil Ethoxylates and Polyethoxylated Castor Oil) refers to a nonionic surfactant having many industrial applications. Polyoxyethylene castor oil derivatives are complex mixtures of various hydrophobic and hydrophilic components. In the polyethoxylated castor oil, the hydrophobic constituents comprise about 80% of the total mixture, the main component being glycerol polyethylene glycol ricinoleate. Other hydrophobic constituents include fatty acid esters of polyethylene glycol along with some unchanged castor oil. The hydrophilic part consists of polyethylene glycols and glycerol ethoxylates.

Further, ethoxylated castor oil is also referred to as a mixture of triricinoleate esters of ethoxylated glycerol with small amounts of polyethyleneglycol (macrogol) ricinoleate and the corresponding free glycols. Polyoxyethylene castor oil derivatives are nonionic surfactants used in oral, topical and parenteral pharmaceutical formulations.

In certain embodiments, the derivative of polyethoxylated castor oil of the compositions of the invention is C:E. As such, the present invention specifically relates to a version of polyethoxylated castor oil known as Cremophor EL or more recently Kolliphor EL (registered trademark of BASF Corp) and also polyoxyethylenglyceroltriricinoleat 35 (DAC), polyoxyl 35 castor oil (USP/NF), obtained by reacting ethylene oxide with castor oil (molar ratio 35:1). The main component of C:E is glycerol-polyethylene glycol ricinoleate, which, together with fatty acid esters of polyethylene glycol, represents the hydrophobic part of the product. The smaller, hydrophilic part consists of polyethylene glycols and ethoxylated glycerol. Due to this particular composition, C:E is capable to stabilize emulsions of nonpolar materials in aqueous solutions, thus making it a universal nonionic emulsifying agent for the pharmaceutical, cosmetic and food industries. Some anti-neoplastic agents (e.g. Taxol, Taxotere) were formulated in C:E and ethanol to enhance drug solubility and therapeutic effect.

When describing the present invention, the terms emulsifying agents, excipient and surfactant are interchangeable.

Specifically, Cremophor EL (CAS Registry number 63393-92-0) (Synonyms Macrogolglycerol ricinoleate, PEG-35 castor oil, Polyoxyl 35 hydrogenated castor oil, Polyoxyl-35 castor oil) denotes a derivative of castor oil or an ester with ethoxylated glycerol of Molecular Formula C₅H₁₂O₄; Molecular Weight: 136.14638 [g/mol]; Formal Charge: 0; Boiling Point 290° C. at 760 mmHg; Flash Point 160° C.

Further, the term C:E designates preparation of Cremophor EL in ethanol (1:1 v/v) and it represents 30% v/v when emulsified in PBS. According to some specific embodiments, the Cremophore EL may be dissolved in or combined with EtOH. More specifically, the C and the E (EtOH) ratio may range between about 1:0 to 1:999999, more specifically, 1:1 to 1:99999, 1:1 to 1:9999, 1:1 to 1:999, 1:1 to 1:99, 1:1 to 1:9. Nevertheless, it should be appreciated that the Cremophor of the invention may be prepared or dissolved in any other solvent.

As noted above, the combined compositions of the invention comprise at least two active agents, specifically, SE and C:E. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

Relying on present findings of particular properties of SE and C:E, in combination or alone, in facilitating control of blood sugar levels and protecting liver function, it is conceived that in specific embodiments the present invention pertains to compositions in a formulation adapted for add-on to a solid, semi-solid or liquid food, beverage, food additive, food supplement, medical food, botanical drug, drug and/or a pharmaceutical compound.

In certain embodiments, the combined composition of the invention may comprise an adjuvant such as any one of polyethylene glycol or beta cyclo dextrin or any derivative thereof. The term “adjuvant” as used herein refers to a pharmacological agent that modifies and enhances the effect of other active agents. It should be noted that the specific adjuvants indicated herein were now surprisingly shown by the invention as exerting a therapeutic effect/s as active main ingredients and not only as additional enhancing or inherent agents.

In some specific embodiments, the combined compositions of the invention may comprise any SE as discussed above and Polyethylene glycol or any derivatives thereof. Polyethylene glycol (PEG) is a polyether compound PEG is also known as polyethylene oxide (PEO) or polyoxyethylene (POE), depending on its molecular weight. PEG, PEO, or POE refers to an oligomer or polymer of ethylene oxide. The three names are chemically synonymous, PEG refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete.

PEG is soluble in water, methanol, ethanol, acetonitrile, benzene, and dichloromethane, and is insoluble in diethyl ether and hexane. It is coupled to hydrophobic molecules to produce nonionic surfactants. When attached to various protein medications, polyethylene glycol allows a slowed clearance of the carried protein from the blood. This makes for a longer-acting medicinal effect and reduces toxicity, and allows longer dosing intervals.

PEG is used as an excipient in many pharmaceutical products. Lower-molecular-weight variants are used as solvents in oral liquids and soft capsules, whereas solid variants are used as ointment bases, tablet binders, film coatings, and lubricants.

In more specific embodiments, The term ‘Polyethylene Glycol’ (CAS Registry number 25322-68-3; CA Index Name: Poly(oxy-1,2-ethanediyl), α-hydro-ω-hydroxy-) denotes an addition polymer of ethylene oxide and water, represented by the formula H(OCH2CH2)nOH, denoted herein as Formula I:

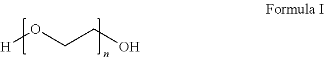

in which n represents the average number of oxyethylene groups. In some embodiments, the average molecular weight is not less than 95.0% and not more than 105.0% of the labeled nominal value if the labeled nominal value is below 1000; it is not less than 90.0% and not more than 110.0% of the labeled nominal value if the labeled nominal value is between 1000 and 7000; it is not less than 87.5% and not more than 112.5% of the labeled nominal value if the labeled nominal value is above 7000. It may contain a suitable antioxidant.

Thus, in certain embodiments, the invention provides a combination of soy extracts or any fractions thereof and PEG for use as described by the invention,

The term PEG designates preparation of PEG in ethanol (1:1 v/v) and it represents 30% v/v when emulsified in PBS. According to some specific embodiments, the PEG may be dissolved in or combined with EtOH. More specifically, the PEG and the E (EtOH) ratio may range between about 1:0 to 1:999999, more specifically, 1:1 to 1:99999, 1:1 to 1:9999, 1:1 to 1:999, 1:1 to 1:99, 1:1 to 1:9. The PEG of the invention may be prepared or dissolved in any other solvent.

As noted above, in some alternative embodiments, the combined compositions of the invention may comprise at least two active agents, specifically, soy extracts and any fractions thereof and PEG. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

Still further alternative embodiments of the invention encompass the provision of combined compositions comprising as active ingredients soy extract and any fractions thereof and Beta cyclo dextrin (BCD).

Cyclodextrins (sometimes called cycloamyloses) are a family of compounds made up of sugar molecules bound together in a ring (cyclic oligosaccharides). Cyclodextrins are produced from starch by means of enzymatic conversion. They are used in food, pharmaceutical, drug delivery, and chemical industries, as well as agriculture and environmental engineering. Cyclodextrins are composed of 5 or more α-D-glucopyranoside units linked 1->4, as in amylose (a fragment of starch). The 5-membered macrocycle is not natural. Recently, the largest well-characterized cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape: α (alpha)-cyclodextrin: 6-membered sugar ring molecule; β (beta)-cyclodextrin: 7-membered sugar ring molecule; γ (gamma)-cyclodextrin: 8-membered sugar ring molecule. α- and γ-cyclodextrin are being used in the food industry. As α-cyclodextrin is a soluble dietary fiber, it can be found as Alpha Cyclodextrin (soluble fiber) on the list of ingredients of commercial products.

Because cyclodextrins are hydrophobic inside and hydrophilic outside, they can form complexes with hydrophobic compounds. Thus they can enhance the solubility and bioavailability of such compounds. This is of high interest for pharmaceutical as well as dietary supplement applications in which hydrophobic compounds shall be delivered. Alpha-, beta-, and gamma-cyclodextrin are all generally recognized as safe by the FDA. In the food industry, cyclodextrins are employed for the preparation of cholesterol free products.

More specifically, the term ‘β-Cyclodextrin’ (CAS Registry number 7585-39-9; Synonyms Cycloheptaamylose, Cyclomaltoheptaose, β-cycloamylose, cycloheptaglucan, cycloheptaglucosan, Betadex) denotes a cyclodextrin composed of seven α-(1→4) linked D-glucopyranose units C₄₂H₇₀O₃₅; Molecular Weight 1134.98 [g/mol]

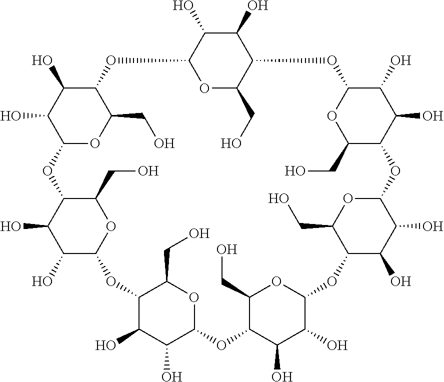 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In more specific embodiments, the invention provides combined compositions comprising as active ingredients SE and Methyl-β-cyclodextrin.

Both β-cyclodextrin and methyl-β-cyclodextrin (MβCD) remove cholesterol from cultured cells. The methylated form MβCD was found to be more efficient than β-cyclodextrin. The water-soluble MβCD is known to form soluble inclusion complexes with cholesterol, thereby enhancing its solubility in aqueous solution. MβCD is employed for the preparation of cholesterol-free products: the bulky and hydrophobic cholesterol molecule is easily lodged inside cyclodextrin rings that are then removed. MβCD is also employed in research to disrupt lipid rafts by removing cholesterol from membranes.

Further, the term BCD designates preparation of BCD in ethanol (1:1 v/v) and it represents 30% v/v when emulsified in PBS. According to some specific embodiments, the BCD may be dissolved in or combined with EtOH. More specifically, the BCD and the E (EtOH) ratio may range between about 1:0 to 1:999999, more specifically, 1:1 to 1:99999, 1:1 to 1:9999, 1:1 to 1:999, 1:1 to 1:99, 1:1 to 1:9.

Nevertheless, it should be appreciated that the BCD of the invention may be prepared or dissolved in any other solvent.

As noted above, the combined compositions of the invention comprise at least two active agents, specifically, soy fractions and BCD. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

Yet in other specific embodiments, the invention provides combined compositions comprising as active ingredients soy fractions and at least one other inactive ingredient that in the present case may work as an active ingredient and synergistically lower blood sugar levels. The term ‘inactive ingredient’, as defined by the US Food and Drug Administration (FDA), refers to an excipient, solvent, binding material or preservative that is generally considered inert or pharmacologically inactive. There are examples however, such as in the case of the present invention, that inactive ingredient in certain combinations and conditions become active ingredients (for examples see further below), this term also encompasses an agent that combines to an active ingredient to facilitate drug transport.

Examples of FDA approved inactive ingredients which are used in drug and food industry and therefore may be applicable to compositions of the present invention include, although not limited to the following ingredients. Thus, in specific embodiments, the present invention may provide a combined composition comprising at least one natural or synthetic soy derivatives and at least one of: acacia, acetic acid, vitamins such as alpha-tocopherol (vitamin E1), ascorbic acid (vitamin C), ascorbyl palmitate (i.e. a fat-soluble form of vitamin C recognized as GRAS), riboflavin (vitamin B2), certain amino acids such as glycine, phenylalanine, and variants thereof such as cysteine hydrochloride, certain salts in particular certain aluminum salts (e.g. aluminum stearate), and sodium, magnesium, potassium and calcium salts (e.g. sodium alginate), benzalkonium chloride, betadex (i.e. beta-cyclodextrin referred to above), butylparaben, Candelilla wax, Carrageenan, castor oil (or a derivative thereof as mentioned above) and other vegetative oils such as coconut, corn, cottonseed, palm kernel, sesame, sunflower, olive, peanut and soybean oils, including hydrogenated variants thereof, cellulose compounds (also microcrystalline cellulose), cetostearyl alcohol including cetyl alcohol, cetylpyridium chloride, citric acid, croscarmellose sodium (also sodium CMC recognized as GRAS), diethyl phthalate (DEP), dimethylaminoethyl methacrylate-butyl methacrylate-methyl methacrylate copolymer (brand name Eudragit EPO) and other Eudragit derivatives, docusate sodium (also dioctyl sodium sulfosuccinate), lanolin, lecithin, ethyl cellulose, fumaric acid, gelatin, glucosamine, glycerin (also glycerol, glycerine), glyceryl behenate, glyceryl distearate, glyceryl monostearate, glyceryl triacetate, isobutylparaben, medium chain triglycerides (MCT), methylparaben, propylparaben and butylparaben, monosodium citrate (recognized as GRAS), poloxamers of common available grades 68, 88, 98, 108, 124, 188, 237, 338, and 407, polycarbophil, polyethylene glycol (PEG referred to above of average molecular weight in the range of 300-8000), polygalacturonic acid, polyplasdones, polysorbates, polyvinylpyrrolidone, povidones, propyl gallate, propylparaben, sorbic acid, succinic acid, tartaric acid, taurine, tragacanth, triacetin and triethyl citrate (recognized as GRAS).

Further inactive ingredients such as sulfites, benzoates (i.e. parabens), benzoic acid, boric acid, bronopololeic acid, butylated hydroxyanisole (BHA, E320), butylated hydroxytoluene (BHT, E321), chlorobutanol, chlorocresol, dimethyl sulfoxide, sorbitan and sorbitan derivatives may be used in certain proportions for the same purpose, as these agents have been reported to cause adverse reactions in some patients. Thus, in some specific embodiments, the invention provide combined compositions comprising an effective amount of at least one natural or synthetic soy extracts or any fractions thereof and at least one of: sulfites, benzoates (i.e. parabens), benzoic acid, boric acid, bronopololeic acid, butylated hydroxyanisole (BHA, E320), butylated hydroxytoluene (BHT, E321), chlorobutanol, chlorocresol, dimethyl sulfoxide, sorbitan and sorbitan derivatives.

It must be appreciated that the combined compositions defined above, as well as any combined composition defined and provided by the invention may be used as add-on to any a solid, semi-solid or liquid food, beverage, food additive, food supplement, medical food, botanical drug, drug and/or any type of pharmaceutical compound.

Still further, the invention further encompasses any soft or an alcoholic beverage comprising at least one natural or synthetic soy extracts and any fractions thereof and at least one of any of the ingredients indicated herein above.

Still further, the invention provides methods as specified by the invention using any of the combined compositions described above or any combinations thereof.

In some embodiments, the add-on composition according to the invention may be formulated as a food additive, food supplement or medical food. In other embodiment, such add-on composition of the invention may be further added or combined with botanical drugs, drugs or any type of pharmaceutical products. The term ‘add-on’ as used herein is meant a composition or compound that may be added to existing compound, composition or material enhancing desired properties thereof or alternatively, adding specific desired property to an existing compound.

More specifically, in certain embodiments, the combined composition of the invention may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement the normal diet. In this sense, this term also encompasses food additives and dietary ingredients. Further, under the Dietary Supplement Health and Education Act of 1994 (DSHEA), a statute of US Federal legislation, the term dietary supplement is defined as a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients.

Under food or dietary supplements is meant those marketed in a form of pills, capsules, powders, drinks, and energy bars and other dose forms. Unlike drugs, however, they are mainly unregulated, i.e. marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet.

In yet some further embodiments, the combined composition of the invention may be an add-on to medical foods. Further in this connection should be mentioned medical foods, which are foods that are specially formulated and intended for the dietary management of a disease that has distinctive nutritional needs that cannot be met by normal diet alone. The term medical food, as defined in the FDA's 1988 Orphan Drug Act Amendments is a food which is formulated to be consumed or administered enterally under the supervision of a physician and which is intended for the specific dietary management of a disease or condition for which distinctive nutritional requirements, based on recognized scientific principles, are established by medical evaluation. Hence, medical foods are subject to the general food and safety labeling requirements of the Federal Food, Drug, and Cosmetic Act. Medical foods are usually classified as nutritionally complete or incomplete formulas, formulas for metabolic disorders and oral rehydration products. Notable examples of the above include gamma-linolenic acid (GLA) and/or a short chain omega-6 fatty acid sourced from the seeds of the borage plant for management of allergic conditions; slowly digested carbohydrates for maintenance of optimal blood sugar levels especially in patients with diabetes; and glutamine for nourishment of the gastrointestinal (GI tract) in metabolically stressed patients.

Also pertinent to the present context are botanical drugs. In specific embodiments, compositions of the invention may be an add-on to a botanical drug. As used herein botanical drug are products that are intended for use in the diagnosis, cure, mitigation, treatment or prevention of disease in humans. A botanical drug product consists of vegetable materials, which may include plant materials, algae, macroscopic fungi, or combinations thereof. A botanical drug product may be available as (but not limited to) a solution (e.g., tea), powder, tablet, capsule, elixir, topical, or injection. Botanical drug products often have unique features, for example, complex mixtures, lack of a distinct active ingredient, and substantial prior human use. Fermentation products and highly purified or chemically modified botanical substances are not considered botanical drug products. According to the FDA Guidance for Industry, a botanical product may be a food (including a dietary supplement), a drug (including a biological drug), a medical device (e.g., gutta-percha), or a cosmetic. Further, botanical drugs may include botanical ingredients in combination with either a synthetic or highly purified drug or a biotechnology derived or other naturally derived drug. In the same way, botanical drugs may also contain animals or animal parts (e.g., insects, annelids, shark cartilage) and/or minerals or a combination thereof.

Specifically pertinent to the present context are foods or food supplements based on soybean (US) or soya bean (UK) or any soy-derived extract.

It is also conceived that for the purpose of specific embodiments and methods, the combined composition of the invention may be an add-on to any type of drugs or therapeutic compounds administered orally, intravenously, intradermaly, by inhalation or intrarectaly. Examples of such combined compositions include, but are not limited to a tissue derived antigens, tumor associated antigens, or viral and or bacterial and or fungal and or parasitic or bacterial derived antigens, or any type of organism derived antigens. It should be further noted that the composition of the invention that comprise any type of SE with or without CE, or of any combination of different SE with or without CE, may be add-on to any type of healthy of diseased tissue derived antigens, or any type of drug or therapeutic compound, or any type of organism derived antigens, or hormones, or cytokines, or antibody, or any type of natural or non-natural compound that may have therapeutic properties. More specifically, such add-on preparation may be used for promoting the effect of said therapeutic compound, for exerting an adjuvant effect, or for improving the therapeutic effect of said drug, compound, or antigen.

Of particular relevance to this context, compositions of the invention as add-on products to hormones, including but not limited to insulin, whether natural or synthetic.

In other specific embodiments, compositions of the invention may be add-on products to at least one gut hormone. Yet in alternative embodiments, the combined composition of the invention may be used as add-on products for concomitant administration of at least one gut hormone. In more particular but non-limiting embodiments, gut hormone include Ghrelin, Cholecystokinin, Cholecystokinin, Peptide YY, Pancreatic polypeptide, Amylin, Glucose-dependent insulinotropic polypeptide, Glucagon-like peptide-1, Glucagon-like peptide-2 and Oxyntomodulin. In more specific embodiments the combined composition of the invention may comprise Ghrelin. As used herein Ghrelin is a peptide hormone released from the stomach and liver and is often referred to as the “hunger hormone” since high levels of it, are found in individuals that are fasting. Ghrelin antagonistic treatments can be used to treat illnesses such as anorexia and loss of appetites in cancer patients. Ghrelin treatments for obesity are still under intense scrutiny and no conclusive evidence has been reached. This hormone stimulates growth hormone release. In yet some further embodiments the combined composition of the invention may further comprise Cholecystokinin. As used herein Cholecystokinin is responsible for gall bladder secretions, gastrointestinal motility as well as pancreatic exocrine secretions. Peptide YY that may be also comprised within the composition of the invention is involved mostly in satiation modulation. Still further, the combined compositions of the invention may comprise Pancreatic polypeptide. Pancreatic polypeptide function is most apparent in control of gastrointestinal motility and satiation. In further embodiments Amylin may be also added to the combined compositions of the invention. Amylin controls glucose homeostasis and gastric motility. Further embodiments relate to the addition of Glucose-dependent insulinotropic polypeptide to the combined compositions of the invention. Glucose-dependent insulinotropic polypeptide possesses an acute influence on food intake through its effects on adipocytes. In further embodiments, Glucagon-like peptide-1 may be added to the compositions of the invention. Glucagon-like peptide-1 has an effect on incretin activity as well as satiation. In other embodiments, Glucagon-like peptide-2 may be added to the compositions of the invention. Glucagon-like peptide-2 is responsible for gastrointestinal motility and growth. Further embodiments relate to the addition of Oxyntomodulin to the combined compositions of the invention. Oxyntomodulin plays a role in controlling acid secretion and satiation.

In yet another embodiment the composition of the invention may be administered as an add-on to a further therapeutic agent that may be an autologous protein-containing tissue extract, for example, colon or liver. Such extract comprises disease-associated antigens that modulate the immune response in the treated subject.

Of particular interest are certain embodiments in which compositions of the present invention are used as add-on to foods and/or beverages comprising an increased content of sugar and/or alcohol or are associated with increase in blood sugar and/or alcohol level.

In a broader sense, compositions of the invention may be adapted for add-on to food and/or beverage that comprise an increased content of sugar and/or alcohol or to a food or beverage that may be associated with increase in blood sugar or alcohol level via alteration of the insulin resistance state or the capability to alter alcohol metabolism by the body.

As previously mentioned, temporary fluctuations of blood glucose levels may develop under various conditions, among which consumption of sugar sweetened or alcoholic beverages represent a significant contributing factor.

In this context, a sugar sweetened beverage (SSB) is any beverage with added sugar, including for example fruit or fruit-flavored drinks, flavored water or sodas, energy drinks (also referred to as soft drinks), as well as coffees, teas and nonalcoholic wines and beers. For the purpose of describing the invention, the terms added sugar, sugar sweetened and high sugar content are interchangeable. Risks of weight gain, obesity and diabetes which have been linked to consumption of sweetened beverages will be discussed further below.

An alcoholic beverage is a drink typically containing 0.1-95% alcohol, most commonly ethanol but occasionally also other alcohols. Alcoholic beverages include beers, wines, and spirits (distilled beverages). For the purpose of the present invention, the term an alcoholic beverage encompasses any kind of alcohol containing beverage produced by process of fermentation or distillation or both, or any type of food or drink that directly or indirectly affect the metabolism of alcohol. Consequences of alcohol consumption, such as alcohol intoxication, hangover and liver damage, well as the link between alcohol consumption and blood sugar levels, are discussed further below.

Basing on present findings of particular properties of compositions of the invention, it is conceived that said compositions as well as all their above described derivatives and combinations are used for controlling blood sugar levels in a subject, wherein said control is inhibiting increase or decrease in blood sugar levels, improving glucose tolerance or altering insulin resistance state.

As meant herein, the terms blood sugar level or blood glucose level imply molar concentration of glucose in the blood or serum of an organism (human or animal) Glucose being, with some exceptions, the primary source of energy for all body's cells, is transported from the intestines or liver to body cells via the bloodstream and is made available for cell absorption via the hormone insulin produced primarily in the pancreas. The body's homeostatic mechanism keeps blood glucose levels within a narrow range by means of several interacting systems, of which hormone regulation is the most important. There are two types of mutually antagonistic metabolic hormones affecting blood glucose levels: (1) catabolic hormones (such as glucagon, cortisol and catecholamines) which increase blood glucose; and (2) an anabolic hormone (insulin) which decreases blood glucose.

Glucose levels are usually lowest in the morning, before the 1^{st} meal of the day (termed the fasting level) and rise after meals for an hour or two by a few millimolars. Blood sugar levels outside the normal range may be an indicator of certain medical conditions. A persistently high level is referred to as hyperglycemia; low levels are referred to as hypoglycemia. Diabetes mellitus is characterized by persistent hyperglycemia from any of several causes, and is the most prominent disease related to failure of blood sugar regulation. Intake of alcohol causes an initial surge in blood sugar, and later tends to cause levels to fall. Certain drugs can also increase or decrease glucose levels.

Blood glucose levels are expressed is in terms of a molar concentration measured in mmol/L (millimoles per litre; or millimolar, abbreviated mM); in the US, blood glucose is measured as mass concentration in mg/dL (milligrams per decilitre). Since the molecular weight of glucose C₆H₁₂O₆ is 180, the difference between the two scales is a factor of 18, so that 1 mmol/L of glucose is equivalent to 18 mg/dL.

One of the important features of the present invention is preventing or reducing temporary fluctuations of blood glucose levels resulting from consumption of sugar-enriched foods and beverages, thus enabling to keep the blood glucose levels within the normal or recommended range.

Under the term normal or recommended blood glucose levels is meant, in humans, the mean normal levels (tested while fasting) are between 70 to 100 mg/dL (3.9 to 5.5 mmol/L) and are restored within this range, if the body's homeostatic mechanism is operating normally. According to the American Diabetes Association, blood sugar levels for those without diabetes and who are not fasting should be below 125 mg/dL. The blood glucose target range for diabetics should be 90-130 mg/dL before meals and less than 180 mg/dL after meals.

According to other estimates, the normal blood glucose level in humans in fasting is approximately 4 mmol/L (4 mM or 72 mg/dL); shortly after a meal the blood glucose level may rise temporarily up to 7.8 mM (140 mg/dL); when operating normally the body restores blood sugar levels to a range of 4.4 to 6.1 mM (82 to 110 mg/dL). For people with type 1 or type 2 diabetes blood sugar level targets are: before meals—4 to 7 mM for; after meals—under 9 mM for people with type 1 and 8.5 mM for people with type 2; children with type 1 diabetes have a greater upper limit for their blood sugar levels by 1 mM.

In this connection, it should be also understood under blood glucose levels is meant arterial, venous and capillary blood glucose levels, which may be comparable or distinct, when fasting or after meals.

Further, the present invention may be applicable in conjunction with measurements or monitoring of blood glucose levels using any available technology, including direct-to-customer glucose blood testing, such as disposable test-strips or electronically-based devices. This is particularly applicable for subjects with diabetes or insulin resistance.

Another application of blood glucose monitoring is a glucose tolerance test, a medical test in which glucose is given and blood samples taken afterward to determine how quickly it is cleared from the blood. This test is usually used to test for diabetes, insulin resistance, and sometimes reactive hypoglycemia and acromegaly, or rarer disorders of carbohydrate metabolism. In the most commonly performed version of the test, an oral glucose tolerance test (OGTT), a standard dose of glucose is ingested by mouth and blood levels are checked two hours later. Many variations of the GTT have been devised over the years for various purposes, with different standard doses of glucose, different routes of administration, different intervals and durations of sampling, and various substances measured in addition to blood glucose. Usually the OGTT is performed in the morning as glucose tolerance can exhibit a diurnal rhythm with a significant decrease in the afternoon. The patient is instructed to fast (water is allowed) for 8-12 hours prior to the tests. The oral glucose challenge test (OGCT) is a short version of the OGTT, used to check pregnant women for signs of Gestational Diabetes. It can be done at any time of day, not on an empty stomach. The test involves 50 g of glucose, with a reading after one hour.

Since the 1970s, the World Health Organization and other organizations interested in diabetes agreed on a standard dose and duration. According to standard OGTT protocol:
A zero time (baseline) blood sample is drawn.
The patient is then given a measured dose of glucose solution to drink within 5 min
Blood is drawn at intervals for measurement of glucose, and sometimes insulin levels.

The intervals and number of samples vary according to the purpose of the test. For simple diabetes screening, the most important sample is the 2 hour sample and the 0 and 2 hour samples may be the only ones collected. A laboratory may continue to collect blood for up to 6 hours depending on the protocol requested by the physician. Fasting plasma glucose (measured before the OGTT begins) should be below 6.1 mmol/L (110 mg/dL). Fasting levels between 6.1 and 7.0 mmol/L (110 and 125 mg/dL) are borderline (“impaired fasting glycaemia”), and fasting levels repeatedly at or above 7.0 mmol/L (126 mg/dL) are diagnostic of diabetes. A 2 hour OGTT glucose level below 7.8 mmol/L (140 mg/dL) is normal, whereas higher glucose levels indicate hyperglycemia. Blood plasma glucose between 7.8 mmol/L (140 mg/dL) and 11.1 mmol/L (200 mg/dL) indicate impaired glucose tolerance and levels above 11.1 mmol/L (200 mg/dL) at 2 hours confirms a diagnosis of diabetes. For the 75 g OGTT: fasting should be below 5.1 mmol/L; 1 hour should be below 10.0 mmol/L; 2 hour should be below 8.5 mmol/L.

Glucose tolerance test is particularly relevant for the diagnosis of insulin resistance state. Insulin resistance describes the body's lack of sensitivity to the hormone insulin, meaning body cells such as the muscle, fat and liver cells are not adequately stimulated to take up glucose from the blood, even when insulin levels are high. This under-utilization of blood glucose results in hyperglycemia or a raised blood sugar level. Tests for diagnosing insulin resistance include:
Fasting blood sugar and postprandial blood sugar—Blood sugar is almost always raised in people with insulin resistance.
Fasting insulin assessment—In a healthy person who has fasted for 6 to 8 hours (usually overnight), the insulin level is approximately 60 pmol/L. A level higher than this is considered indicative of insulin resistance.
Glucose tolerance testing (GTT)—For a glucose tolerance test, a person fasts for 8 to 12 hours (usually overnight) and is then given a 75 gram oral dose of glucose. After two hours, the blood levels of glucose are measured.
In a healthy person, the blood sugar level after two hours is usually less than 7.8 mmol/L (140 mg/dl). A blood sugar level between 7.8 and 11.0 mmol/dl (140 to 197 mg/dl), however, indicates impaired glucose tolerance. If the level is over 11.1 mmol/dl (200 mg/dl), diabetes mellitus is diagnosed.
Modified insulin suppression test—For this test, patients are given 25 mcg of octreotide (an inhibitor of insulin and glucagon) over 3 to 5 minutes and are then infused with somatostatin (0.27 μgm/m2/min) to suppress the release of insulin and glucose into the blood.

Further, the terms preventing, reducing or controlling fluctuations of blood glucose levels or glucose tolerance levels or insulin resistance state levels are meant to convey preventing, reducing or controlling increase as well as decrease in blood sugar levels, i.e. increase or decrease of at least about 0.1% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or 60%.

In more specific embodiments, the compositions of the invention may attenuate, decrease, inhibit, prevent, reduce or minimize the increase or elevation in blood sugar levels caused by high sugar beverages or foods in at least about 0.1% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% or more, specifically, 65%, 70%, 75%, 80%, 85%, 90%, 95% or more.

As previously mentioned, temporary fluctuations of blood glucose levels may develop under various conditions, among which consumption of sugar sweetened or alcoholic beverages represent a significant contributing factor. In addition, alteration of blood sugar levels can occur following use of medications or in other states altering the level of insulin resistance.

Alcohol interferes with all three sources of glucose and hormones needed to maintain healthy blood glucose levels. The greatest impact is seen in those who drink heavily and on frequent basis. In heavy drinkers, glycogen stores are depleted within few hours, if their diet does not provide a sufficient amount of carbohydrates. Over time, excessive alcohol consumption can decrease insulin's effectiveness, resulting in high blood sugar levels; according to certain estimates 45% to 70% of people with alcoholic liver disease had either glucose intolerance or diabetes.

Alcohol can also negatively impact blood sugar levels each time that it is consumed, regardless of the frequency of consumption. Research has shown that acute consumption increases insulin secretion causing low blood sugar (hypoglycemia) and leading to impairment of hormonal responses that would normally rectify the low blood sugar. Drinking as little as 2 ounces of alcohol on an empty stomach can lead to very low blood sugar levels. This makes alcohol an even bigger problem for people with diabetes. Along with the impact on blood sugar, studies have also shown that alcohol can impact the effectiveness of the hypoglycemic medications, so extreme caution needs to be taken when consuming alcohol by anyone with diabetes.

There is also an increased risk of problems when combining exercise and alcohol. While blood sugars naturally drop during exercise and a body is working on replacing its glycogen stores, consuming alcohol during this time will halt this process and can cause blood sugar levels to stay at an unhealthy level.

The present meaning of alcohol consumption encompasses the entire range of associated physiological, psychological, social conditions, i.e. social drinking, session drinking, binge drinking alcohol abuse, alcohol intoxication and alcoholism. Meaning of these terms in the present context is detailed below.

Further, it is conceived that compositions of the present invention are used for prevention or alleviation of symptoms related to a condition associated with increased or decreased blood sugar levels, wherein said condition is any one of diabetes, obesity, hepatic disorder, pancreatic dysfunction, weight gain, alcohol intoxication, alcohol withdrawal and vertigo, any condition associated with alteration of pancreatic or liver function or tissue or organ damage.

It should be therefore understood that compositions of the present invention are particularly applicable to clinical as well as sub-clinical conditions associated with increased or decreased blood sugar levels. Clinical applications of the present invention, specifically to diabetes, obesity, hepatic disorder, pancreatic dysfunction and insulin resistance, will be detailed further below. Applications to sub-clinical conditions which are more common, such as weight gain, alcohol intoxication, alcohol withdrawal, vertigo and any condition associated with alteration of pancreatic or liver function or tissue or organ damage, are presently discussed.

In this context, “weight gain” is meant an increase in body weight, particularly by way of increased body fat deposits (adipose tissue), than is optimally healthy. A person generally gains fat-related weight by increasing food consumption or by becoming physically inactive, or by both. When energy intake exceeds energy expenditure, the body stores the excess energy in a dense high-energy form as fat. One pound of fat stores 3500 calories of energy, so over time, excessive energy intake and/or lack of exercise can contribute to fat gain and obesity. Having excess fat is a common condition, as much as 64% of the US adult population is considered either overweight or obese, and this percentage has increased over the last four decades. Weight gain has a latency period. The effect that eating has on weight gain can vary greatly depending on the following factors: energy (calorie) density of foods, exercise regimen, amount of water intake, amount of salt contained in the food, time of day eaten, age of individual, individual's country of origin, individual's overall stress level and amount of water retention in ankles/feet. Typical latency periods vary from three days to two weeks after ingestion. Weight gain is also a common side-effect of certain psychiatric medications. Weight gain is seen in certain professional sports.

In this connection, the present invention is relevant to prevention of weight gain in all its measurements and forms. One of the ways to assess abnormal weight is by the measurement of Body Mass Index (BMI), or Quetelet index, which is a measure of relative weight based on an individual's mass and height. The WHO regards a BMI of less than 18.5 as underweight indicative of malnutrition, an eating disorder or other health problems, while a BMI greater than 25 is considered overweight and above 30 is considered obese.

Increase in body fat percentage or an excess of adipose tissue on a human can lead to serious health side-effects. A large number of medical conditions have been associated with obesity. Health consequences are categorized as being the result of either increased fat mass (osteoarthritis, obstructive sleep apnea, social stigma) or increased number of fat cells (diabetes, some forms of cancer, cardiovascular disease, non-alcoholic fatty liver disease). There are alterations in the body's response to insulin (insulin resistance), a proinflammatory state and an increased tendency to thrombosis (prothrombotic state). The ever-present social stigma concerning weight gain can have lasting and harmful effects, especially among young women.

Thus, the present invention being applicable to prevention of weight gain is thereby also applicable to prevention or reduction of risk for all the above mentioned conditions.

In certain embodiments, compositions of the present invention are particularly applicable to prevention and reduction of symptoms of alcohol intoxication, alcohol withdrawal and vertigo. Symptoms of alcohol intoxication include reduced activity in the central nervous system (CNS), loose muscle tone, loss of fine motor coordination, a staggering “drunken” gait, eyes appear “glossy,” pupils may be slow to respond to stimulus, pupils may become constricted, decreased heart rate, lower blood pressure and respiration rate, decreased reflex responses, slower reaction times, skin may be cool to the touch, profuse sweating, loss of fine motor coordination, or odor of alcohol on the breath. Diagnostic criteria for alcohol intoxication are detailed in the Diagnostic and Statistical Manual of Mental Disorders, Fourth Edition (DSM-IV).

The term alcohol intoxication as used herein refers to a situation where the quantity of alcohol a person consumes exceeds the individual's tolerance for alcohol and thus produces, either during or shortly after drinking, clinically important psychological, behavioral or physical abnormalities, such as inappropriate aggression, and impaired judgment and social functioning. One or more of the following signs or symptoms of alcohol intoxication occur shortly after drinking: (1) slurred speech; (2) impaired motor coordination; (3) unsteady gait; (4) nystagmus (involuntary, irregular eye movement characterized by smooth pursuit of an object in one direction and saccadic movement in the other direction); (5) inattention and/or impaired memory; and (6) stupor or coma.

Sobriety, intoxication, alcohol abuse, alcohol-related aggression or alcoholism may be measured according to one or more recognized tests, such as psychomotor tests, serum alcohol level tests, for example accepted inhalation tests, or according to DSM-IV, Alcohol Abstinence Self-Efficacy Scale, Barratt Impulsiveness Scale—11, State-Trait Anger Expression Inventory—2, Conflict Resolution, Impulsivity and Aggression Questionnaire, Social Problem-Solving Inventory—Revised, Alcohol-Related Aggression Questionnaire, or The Alcohol Use Disorders Identification Test. Levels of alcohol in the body may be measured in urine, blood, breath or saliva.

There is a wide range of variability in blood alcohol levels that different individuals can tolerate without becoming intoxicated. The range may be as great as from 0.3 to 1.5 mg/ml, although most states in the U.S. set the sobriety level for legally driving at 0.8 mg/ml. Some users may develop significant behavioral changes or become intoxicated at a much lower Blood Alcohol Concentration (BAC) than the legal limit. This condition is known as “Alcohol Idiosyncratic Intoxication” or “Pathological Intoxication”. In general:
0.02-0.03 BAC no loss of coordination, slight euphoria and loss of shyness.
0.04-0.06 BAC feeling of well-being, relaxation, lower inhibitions, sensation of warmth, euphoria, some minor impairment of reasoning and memory.
0.07-0.09 BAC slight impairment of balance, speech, vision, reaction time; reduction of judgment and self-control and caution, reason and memory.
0.10-0.125 BAC significant impairment of motor coordination and loss of good judgment; slurred speech; balance, vision, reaction time and hearing are impaired; euphoria. It is illegal to operate a motor vehicle at this level.
0.13-0.15 BAC gross motor impairment and lack of physical control; blurred vision and major loss of balance; euphoria is reduced and dysphoria (anxiety, restlessness) is beginning to appear.
0.16-0.20 BAC dysphoria predominates, nausea may appear.
0.25 BAC the drinker needs assistance in walking; total mental confusion.
0.30 BAC loss of consciousness.
0.40 BAC and up onset of coma, possible death due to respiratory arrest.

The term social drinking refers to the consumption of alcohol in a safe, legal and socially acceptable manner usually without the intent of reaching the point of becoming intoxicated (i.e., to achieve alcohol intoxication). Although the amount of blood alcohol which leads to intoxication varies widely between individuals, three or fewer measured drinks (or a blood alcohol level of up to 0.05%) are generally considered to be within the social drinking range.

The term session drinking refers to drinking in large quantities over a single period of time, without the intention of getting heavily intoxicated. The focus is on the social aspects of the occasion.

The term binge drinking refers to drinking alcohol solely for the purpose of intoxication, although it is quite common for binge drinking to apply to a social situation, creating some overlap in social and binge drinking.

The term alcoholism refers to a primary chronic disease known as alcohol dependence syndrome, the most severe stage of a group of drinking problems. Alcoholism is considered a progressive disease, meaning that the symptoms and effects of drinking alcohol become increasingly more severe over time.

The term alcohol abuse refers to repeated drinking despite alcohol-related physical, social, psychological, or occupational problems (according to DSM-IV). When alcohol abuse reaches the alcohol dependence stage, a person may also experience tolerance, withdrawal, and an uncontrolled drive to drink.

In the context of the present invention, after-effects of alcohol consumption, specifically alcohol hangover, alcohol withdrawal or detoxification are also included, as well as any effect of the alcohol on target organs such as the liver, heart, kidney, brain, muscles, gastrointestinal tract, and any other tissue or organ that can be affected by alcohol or by compounds or states in which the metabolism of alcohol is disturbed.

Alcohol hangover refers to physical and mental symptoms that occur within several hours after alcohol consumption, when a person's BAC is falling, and may continue for up to 24 hours thereafter. Alcohol directly promotes hangover symptoms through its effects on urine production, the gastrointestinal tract, blood sugar concentrations (i.e. hypoglycemia), sleep patterns, and biological rhythms. In addition, effects related to alcohol absence after a drinking bout (i.e., withdrawal), alcohol metabolism, and other factors (e.g., biologically active, non-alcohol compounds in beverages, use of other drugs, certain personality traits and a family history of alcoholism) also may contribute to the hangover condition. The particular set of symptoms experienced and their intensity may vary from person to person and from occasion to occasion. In addition, hangover characteristics may depend on the type of alcoholic beverage consumed and the amount a person drinks.

Physical symptoms of a hangover include fatigue, headache, increased sensitivity to light and sound, redness of the eyes, muscle aches, and thirst. Signs of increased sympathetic nervous system activity can accompany a hangover, including increased systolic blood pressure, rapid heartbeat (i.e., tachycardia), tremor, and sweating. Mental symptoms include dizziness, sense of the room spinning (i.e., vertigo), and possible cognitive and mood disturbances, especially depression, anxiety, and irritability.

Alcohol-induced hypoglycemia generally occurs after binge drinking over several days in alcoholics who have not been eating. In such a situation, prolonged alcohol consumption, coupled with poor nutritional intake, not only decreases glucose production but also exhausts the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Several lines of evidence suggest that a hangover and mild alcohol withdrawal (AW) share a common biological mechanism. 1^{st}, the signs and symptoms of hangover and mild AW overlap considerably. 2^{nd} it has been known that alcohol re-administration alleviates the unpleasantness of both AW syndrome and hangovers.

In further embodiments, compositions of the invention may be applicable for AW and AW syndrome. The AW or AW syndrome or alcohol detoxification, the terms used herein interchangeably, refers to the state following the cessation of excessive drinking, which results from compensatory changes in the CNS that take place in response to chronically administered depressant substances (in this case, alcohol, or more specifically, ethanol). These changes include alterations in the GABA and glutamate receptors, the two main neurotransmitters responsible for inhibitory and excitatory effects. Following chronic alcohol exposure, in an effort to counterbalance the alcohol's sedative effects, the body decreases the number or sensitivity of GABA receptors and increases the number or sensitivity of glutamate receptors. When alcohol is removed from the body, the CNS and a portion of the sympathetic nervous system that coordinates response to stress remain in an unbalanced “overdrive” state. Sympathetic nervous system hyperactivity accounts for the tremors, sweating, and tachycardia observed in both hangover and AW syndrome.

In still further embodiments, compositions of the present invention are applicable to prevention of vertigo, i.e. a subtype of dizziness in which a patient inappropriately experiences the perception of motion (usually a spinning motion) due to dysfunction of the vestibular system. It is often associated with nausea and vomiting as well as a balance disorder, causing difficulties with standing or walking Dizziness and vertigo are common and affect approximately 20%-30% of the general population, they can occur in people of all ages, in women more than in men. Apart from physiological causes of vertigo, such as infections of the inner ear, concussion, migraine, epilepsy and others, excessive drinking of alcohol can also cause symptoms of vertigo.

In yet other embodiments, compositions of the present invention are applicable to prevention of any organ or tissue damage resulting from abnormal alterations of blood sugar or alcohol levels or of the state of insulin resistance in a subject.

The tissue-damaging effects of hyperglycemia are well known in diabetic patients, including microvascular complications (retinopathy and nephropathy), macrovascular complications (ischaemic heart disease, vascular disease, stroke and renal artery stenosis) and neuropathies. Microvascular tissue damage is the results of hyperglycaemia per se. Macrovascular complications are found to be associated with insulin-resistant states and hyperinsulinemia. Due to these complications diabetes is also a most frequent cause of blindness and cardiovascular disease. Certain cells types are known to be vulnerable to direct damage from chronic hyperglycemia, for e.g. mesangial cells of kidney, vascular endothelial cells, pancreatic beta cells, Schwann cells and neurons.

Alcohol affects virtually every organ and tissue in the body, with multi-factorial actions on cellular and molecular functions. Alcohol itself alters biological function by direct interaction with cellular components and also due to effect of alcohol metabolism on the systemic oxidative and inflammatory state. Alcohol metabolism produces acetaldehyde and reactive oxygen (and other) species, biochemical moieties that damage healthy tissue. Oxidative stress ensuing from these reactive oxygen and nitrogen species in many organs and tissues may vary in severity depending on the systemic inflammatory and oxidative state, and on systemic and local immune function.

In specific embodiments, compositions of the present invention are particularly applicable to prevent liver and/or pancreatic tissue damage. Hazardous effects of alcohol on progressive and irreversible damage of the pancreatic (chronic pancreatitis) and liver (liver cirrhosis) tissues are well documented. There is an increased incidence of cirrhosis in diabetic patients, 80% of which have glucose intolerance.

Further, obstruction of pancreatic and liver damage by compositions of the present invention is particularly important for maintenance of glucose homeostasis, the liver being the major organ for insulin-mediated glycogen storage and the pancreas—for production of insulin and glucagon. In this sense, compositions of the present invention are intended to prevent any condition associated with alteration of pancreatic or liver function or alteration of pancreatic or liver metabolic capacity. Those conditions may include drug-induced pancreatic and liver damage, inflammatory pancreatic and liver damage resulting from infections and autoimmune disorders, pancreatic and liver malignancies and other pancreatic and liver dysfunctions.

Still further, compositions of the present invention may be used for prevention of any target organ damage related to conditions associated with abnormal glucose homeostasis, such as hepatic disorders, pancreatic dysfunction, diabetes, obesity, insulin resistance, metabolic disorders or any type of inflammation of the pancreas, liver, muscle or the adipose tissue.

Yet, in another aspect, compositions of the present invention form basis for a pharmaceutical composition for use in a method for treating a subject suffering from a disorder associated with increased or decreased blood sugar levels.

According to certain specific embodiments, compositions of the invention are particularly suitable for oral or mucosal administration. More specifically, oral or mucosal pharmaceutical compositions of the invention are made by combining a therapeutically effective amount of at least one natural or synthetic SE and at least one polyethoxylated castor oil or any derivative thereof, and optionally at least one additional therapeutic agent, with a pharmaceutically acceptable carrier.

The usefulness of an oral formulation requires that the active agent or combinations thereof according to the invention are bioavailable. Bioavailability of orally administered drugs can be affected by a number of factors, such as drug absorption throughout the gastrointestinal tract, stability of the drug in the gastrointestinal tract, and the first pass effect.

Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home. Solid oral dosage forms include, but are not limited to, tablets (e.g., chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release. In addition, a capsule comprising the powder, pellets or granules can be further coated. A tablet or caplet can be scored to facilitate division for ease in adjusting dosage as needed.

As one example, a tablet can be prepared by compression or by molding. Compressed tablets can be prepared, e.g., by compressing, in a suitable machine, the active ingredients (e.g., combined SE and C:E) in a free-flowing form such as powder or granules, optionally mixed with an excipient. Molded tablets can be made, e.g., by molding, in a suitable machine, a mixture of the powdered combined SE and C:E compound moistened, e.g., with no inert liquid diluent.

Liquid dosage forms also allow subjects to easily take the required dose of active ingredient. Liquid preparations can be prepared as a drink, or to be administered, for example, by a naso gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid degradation, decomposition or deactivation of the active agents by the gastrointestinal system, e.g., by gastric fluid in the stomach. For example, compositions can optionally be formulated to pass through the stomach unaltered and to dissolve in the intestines, i.e., enteric coated compositions.

As indicated above, the combined composition of SE and/or polyethoxylated castor oils, specifically C:E, as described herein, can be incorporated into a pharmaceutical composition suitable for oral or mucosal administration, e.g., by ingestion, inhalation, or absorption, e. g., via nasal, intranasal, pulmonary, buccal, sublingual, rectal, dermal, or vaginal administration. Such compositions can include an inert diluent or an edible carrier. For the purpose of oral therapeutic administration, the active C:E and SE compounds can be incorporated with recipients and used in solid or liquid (including gel) form. Oral compositions can also be prepared using an excipient. Pharmaceutically compatible binding agents, and/or adjuvant materials can be included as part of the composition. Oral dosage forms comprising combined SE and C:E are provided, wherein the dosage forms, upon oral administration, provide a therapeutically effective blood level of the combined SE and C:E to a subject. Also provided are mucosal dosage forms comprising said combination wherein the dosage forms, upon mucosal administration, provide a therapeutically effective blood level of the combined SE and C:E to a subject. For the purpose of mucosal therapeutic administration, the active combined compounds (e.g. SE with C:E) can be incorporated with excipients or carriers suitable for administration by inhalation or absorption, e.g., via nasal sprays or drops, or rectal or vaginal suppositories.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art. Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents. Tablets and capsules represent convenient pharmaceutical compositions and oral dosage forms, in which case solid excipients are employed. If desired, tablets can be coated by standard aqueous or non-aqueous techniques. Such dosage forms can be prepared by any of the pharmaceutical methods known in the art. In general, pharmaceutical compositions and dosage forms are prepared by uniformly and intimately admixing the active ingredients with liquid carriers, finely divided solid carriers, or both, and then shaping the product into the desired presentation if necessary.

Although preferred administration is oral or mucosal, it should be appreciated that compositions of the invention may be also suitable for intravenous, intramuscular, subcutaneous, intraperitoneal, perenteral, transdermal, sublingual, topical administration, or any combination thereof.

In some embodiments the above pharmaceutical compositions, in their various formulations, are particularly applicable to treatment of certain clinical disorders associated with alcohol consumption.

It is thus conceived that the above pharmaceutical compositions, in their various formulations, are particularly applicable to treatment of certain clinical disorders, including a hepatic disorder, pancreatic dysfunction, diabetes, obesity, insulin resistance, metabolic syndrome, alcohol intoxication, alcohol withdrawal and vertigo, an inflammation of pancreas, liver, muscle or the adipose tissue, and conditions related thereto.

Specific pharmaceutical compositions comprising a therapeutically effective amount of at least one soy derived polar fraction and at least one polyethoxylated castor oil, any derivative thereof, or any combination thereof are particularly applicable to methods for treating, preventing, ameliorating, reducing or delaying the onset of an immune-related disorder.

In specific embodiments, therapeutic applications of pharmaceutical compositions of the invention include an inflammatory disorder, an autoimmune disorder, an infectious disease and a proliferative disorder.

Detailed discussion on clinical conditions which can be treated or prevented by pharmaceutical compositions of the present invention is presented further below. At this point, it should be understood that, in general, for the purpose of therapeutic applications pharmaceutical compositions are administered in an amount sufficient to cure or at least partially arrest, ameliorate, reduce or delay the onset of symptoms of a clinical condition and its complications, referred to herein as a therapeutically effective amount or dose. Amounts effective for this use will depend upon severity of the condition and the general state of a patient. Single or multiple administrations on a daily, weekly or monthly schedule can be carried out with dose levels and pattern being selected by the treating physician.

Further, it is conceived that for the purpose of specific therapeutic applications, pharmaceutical compositions of the present invention further comprise at least one additional therapeutic agent. More specifically, such agent may be any one of insulin, antibodies directed to inflammatory cytokine, or antibodies such as anti TNF antibodies, statins, analgesics, chemotherapeutic agents and antibiotics.

In further embodiments, pharmaceutical compositions of the invention may optionally further comprise additional therapeutic agent, wherein said additional therapeutic agent is any one of or any type of an organism-derived antigen, including viral and or bacterial and/or fungal and/or parasitic antigens, such as any type of hepatitis B or hepatitis C derived antigens, or any type of bacterial antigens. In further embodiments, compositions of the invention may optionally further comprise additional therapeutic agent, wherein said additional therapeutic agent may be any one of autologous or allogeneic tissue derived proteins, antigens, any type of tissue derived material obtained either from the same or from different species. In further embodiments said tissue derived material or preparations may be obtained from a healthy or diseased tissue. Non-limiting examples include tumor associated tissues, blood products, tissues obtained from an individual infected with a viral or bacterial pathogen that may be combined with any variant or derivative of SE and/or CE, as described above. It is also conceived that for the purpose of specific embodiments of the compositions and methods, the combined composition of the invention may be an add-on to any type of drugs or therapeutic compounds administered orally, intravenously, intradermaly, by inhalation or intrarectaly. These combinations can be used for promoting the effect of any of the above said compounds, or for exerting an adjuvant effect, or for improving the therapeutic effect of said drug, compound, or antigen.

More specifically, pharmaceutical compositions of the invention may optionally further comprise at least one additional therapeutic agent, said additional therapeutic agent is any one of insulin, N-acetyl cysteine (NAC), thiamine (vitamin B1), a benzodiazepine or any combination thereof and a tissue derived preparation or compound.

Insulin is a peptide hormone produced by pancreatic β-cells and is central to regulating carbohydrate and fat metabolism in the body. It causes cells in the skeletal muscles and fat tissue to absorb glucose from the blood. In other words, insulin is an anabolic hormone causing cells to take up energy substrates at the times of excess. Insulin acts through a complex mechanism involving protein phosphorylation and dephosphorylation, which lead to controlled activation of glycogen synthetase and pyruvate dehydrogenase and inactivation phosphofructokinase II and hormone-sensitive lipase. Complicated control mechanism steer hormone secretion such that metabolism is constantly adjusted by hormones to meet our widely varying energy intake and expenditure, assuring a constant internal milieu. Insulin action is countered by the catabolic hormones glucagon, adrenalin, noradrenalin and growth hormone, which act primarily through cyclic AMP (cAMP) and protein kinase A.

Supplementation of exogenous insulin (most commonly injected subcutaneously) is the predominant therapy for patients with type 1 diabetes (which do not produce insulin). Medical preparations of insulin (from the major suppliers—Eli Lilly, Novo Nordisk, and Sanofi Aventis, or others) are specially prepared mixtures of insulin plus other substances including preservatives, which delay absorption of insulin, adjust the pH of the solution to reduce reactions at the injection site. Most of the medical insulin produced today is recombinant insulin, which almost completely replaced insulin obtained from animal sources (e.g. pigs and cattle). A variety of different recombinant human insulin preparations are in widespread use. Since 2003, yeast-based insulin also became available for medical use. In addition, a number of insulin analogues, which retain the hormone's glucose management functionality, have been developed. They are either absorbed rapidly in an attempt to mimic the real β-cell insulin (as with Lilly's lispro, Novo Nordisk's aspart and Sanofi Aventis' glulisine), or steadily absorbed after injection instead of having a ‘peak’ followed by a more or less rapid decline in insulin action (as with Novo Nordisk's version Insulin detemir and Sanofi Aventis's Insulin glargine), all while retaining insulin's glucose-lowering action in the human body.

The major problem with management of insulin therapy is choosing the most appropriate insulin type and dosage/timing for each diabetic patient. The commonly used types are:
fast-acting using insulin analogues aspart, lispro, and glulisine, which begin to work within 5 to 15 minutes and are active for 3 to 4 hours.
short-acting using regular insulin which begins working within 30 minutes and is active about 5 to 8 hours.
intermediate-acting using NPH insulin which begins working in 1 to 3 hours and is active 16 to 24 hours.
long acting using analogues glargine and detemir, each of which begins working within 1 to 2 hours and continue to be active, without major peaks or dips, for about 24 hours.
ultra-long acting currently only including the analogue degludec, which begins working within 30-90 minutes, and continues to be active for greater than 24 hours.
combination insulin products using either fast-acting or short-acting insulin with a longer acting insulin, typically an NPH insulin.

It must be understood that the invention encompasses the use of any insulin preparation as an additional therapeutic agent in any of the pharmaceutical compositions described herein.

Oral, intradermal, intrarectal, inhaled, intrapulmonary, or intramucosl administration of insulin or of compounds that alter insulin metabolism or that alter or potentiate its effects, whether via direct effect following systemic absorption or indirect effect following an effect on the gut associated lymphoid tissue, or any subset of cells with which they are in direct contact, can exert beneficial effect on glucose metabolism. It also has beneficial effect on the metabolic syndrome targets, such as fatty liver disease, NASH, atherosclerosis, heart disease, hyperlipidemia and diabetes.

Still further, in certain embodiments, said additional therapeutic agent may be NAC, N-acetyl cysteine (Brand names: NAC, Mucomyst, Acetadote), which has many uses in medicine. NAC is used to counteract acetaminophen (Tylenol) and carbon monoxide poisoning. It is also used for chest pain (unstable angina), bile duct blockage in infants, amyotrophic lateral sclerosis (ALS, Lou Gehrig's disease), Alzheimer's disease, allergic reactions to the anti-seizure drug phenytoin (Dilantin), and an eye infection called keratoconjunctivitis. It is also used for reducing levels of a type of cholesterol called lipoprotein (a), homocysteine levels (a possible risk factor for heart disease) and the risk of heart attack and stroke in patients with serious kidney disease. Some people use NAC for chronic bronchitis, chronic obstructive pulmonary disease (COPD), hay fever, a lung condition called fibrosing alveolitis, head and neck cancer, and lung cancer. It is also used for treating some forms of epilepsy; ear infections; complications of kidney dialysis; chronic fatigue syndrome (CFS); an autoimmune disorder called Sjogren's syndrome; preventing sports injury complications; radiation treatment; increasing immunity to flu and H1N1 (swine) flu; and for detoxifying heavy metals such as mercury, lead, and cadmium.

Specifically relevant to the present context, NAC is also used for preventing alcoholic liver damage, for protecting against environmental pollutants including carbon monoxide, chloroform, urethanes and certain herbicides; for reducing toxicity of ifosfamide and doxorubicin, drugs that are used for cancer treatment; as a hangover remedy; for preventing kidney damage due to certain X-ray dyes; and for human immunodeficiency virus (HIV).

Healthcare providers give NAC intravenously (IV) for acetaminophen overdose, acrylonitrile poisoning, amyotrophic lateral sclerosis (ALS, Lou Gehrig's disease), kidney failure in the presence of liver disease (hepatorenal syndrome), chest pain in combination with nitroglycerin, heart attack in combination with nitroglycerin and streptokinase, and for helping to prevent multi-organ failure leading to death. NAC is sometimes inhaled or delivered through a tube in the throat to treat certain lung disorders such as pneumonia, bronchitis, emphysema, cystic fibrosis, and others.

Benzodiazepines (sometimes colloquially benzo, often abbreviated BZD), are another class of relevant therapeutic agents. BZD are psychoactive drugs whose core chemical structure is the fusion of a benzene ring and a diazepine ring, the most notable example of which is Valium. BZD enhance the effect of the neurotransmitter GABA at the GABAA receptor, resulting in sedative, hypnotic (sleep-inducing), anxiolytic (anti-anxiety), euphoric, anticonvulsant, and muscle relaxant properties; also seen in the applied pharmacology of high doses of many shorter-acting BZD are amnesic-dissociative actions. These properties make BZD useful in treating anxiety, insomnia, agitation, seizures, muscle spasms, AW and as a premedication for medical or dental procedures.

Still further, in certain embodiments, the additional therapeutic agent may bean immuno-modulatory antibody being administered orally, intravenously, intrarectaly, by inhalation or intradermally. Such antibodies may include, but are not limited to, anti TNF antibodies, both chimeric or humanized, anti integrin antibodies, or any type of antibody. These antibodies may be combined with the combined composition of the invention and/or with any of the above compounds for prevention or amelioration of toxicity or unwanted side effects of sugar, alcohol or any drug. Alternatively, these antibodies may be combined with the compositions of the invention and/or any of the compounds described above for augmenting the beneficial effects of these antibodies or of any of the compounds described herein above.

In yet other embodiments, an additional therapeutic agent may be Vitamin B1. Vitamin B1 (also thiamine or thiamin, i.e. sulfur-containing vitamin) is a water-soluble vitamin of the B complex. Its phosphate derivatives are involved in many cellular processes. The best-characterized form is thiamine pyrophosphate (TPP), a coenzyme in the catabolism of sugars and amino acids. Thiamine is used in the biosynthesis of the neurotransmitter acetylcholine and gamma-aminobutyric acid (GABA). Vitamin B is synthesized only in bacteria, fungi, and plants, animals must obtain it from their diet, and thus, for them, it is an essential nutrient. In mammals, deficiency results in Korsakoff's syndrome, optic neuropathy and Beriberi disease that affects the peripheral nervous system (polyneuritis) and/or the cardiovascular system. Thiamine deficiency has a potentially fatal outcome if it remains untreated. In less severe cases, nonspecific signs include malaise, weight loss, irritability and confusion.

Specifically in this context, alcoholics may have thiamine deficiency due to:
inadequate nutritional intake.
active transport of thiamine into enterocytes is disturbed during acute alcohol exposure.
liver thiamine stores are reduced due to hepatic steatosis or fibrosis.
impaired thiamine utilization due to chronic alcohol consumption.
ethanol per se inhibits thiamine transport in the gastrointestinal system.

Vitamin B1 supplementation is one of the therapeutic approaches to AW syndrome. Following improved nutrition and the removal of alcohol consumption, certain impairments linked with thiamine deficiency are reversed, in particular poor brain functionality.

Thus it is further conceived that certain compositions of the present invention, particularly those comprising a therapeutically effective amount of at least one soy derived polar fraction and at least one polyethoxylated castor oil or any derivative thereof, or any combination thereof can be used in a method for treating liver damage and/or restoring liver function in a subject in need thereof.

More specifically, said compositions are applicable to liver diseases which is any one of viral, bacterial, fungal or parasitic liver disease, alcoholic or autoimmune hepatitis, alcoholic or autoimmune cirrhosis, alcoholic fatty liver disease, non alcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

Still further embodiments of the invention relate to the composition described above for use in a method for treating, preventing, ameliorating, reducing or delaying the onset of acute or chronic toxic effect of alcohol consumption and for restoring liver function.

Further, relying on the exemplified protective effects of compositions of the invention, it is conceived that these compositions will form basis for preparation of “safe drugs”. Particularly, the combined compositions comprising SE and/or C:E and any type of therapeutic compound or food, or any ingredient will provide protection against any type of toxicity or side effect of said drugs, and against any type of target organ toxicity. In addition, such combined compositions may enhance and augment additively or synergistically, the effects of drugs or compounds. These beneficial effects may act via augmenting of the mechanism of action of or via an indirect adjuvant effect, for example by activating other pathways, cells or organs.

Thus, compositions of the invention are also applicable to methods for treating, preventing, ameliorating, reducing or delaying the onset of acute or chronic toxic effect of a drug on any body organ and for restoring liver function wherein the drug induces liver injury. For these purposes compositions of the invention may be administered concomitantly or simultaneously, the latter also include administrations in the same formulation.

More specifically, the present invention further provides pharmaceutical compositions for treating, preventing, ameliorating, reducing or delaying the onset of acute or chronic toxic effect of an analgesic or an antipyretic drug in a subject in need thereof. Moreover, pharmaceutical compositions of the invention may be used for treating and preventing any type of liver insult selected from infectious metabolic, toxic, immune, or perfusion or blood flow related hepatic injury. Pharmaceutical compositions of the invention may comprise as an active ingredient a therapeutically effective amount of a combination of at least one natural or synthetic SE and at least one polyethoxylated castor oil or any derivatives thereof, specifically, C:E, and/or optionally, at least one adjuvant selected from polyethylene glycol and beta cyclo dextrin or any derivative thereof and optionally at least one additional therapeutic agent, with a pharmaceutically acceptable carrier.

In more specific embodiments said therapeutic agent may be an analgesic or an antipyretic drug, such as for example an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to one specific embodiment, pharmaceutical compositions of the invention are intended for treating, preventing, ameliorating, reducing or delaying the onset of acute or chronic toxic effect of the analgesic drug N-(4-hydroxyphenyl) ethanamide, known as acetaminophen (paracetamol).

N-(4-hydroxyphenyl) ethanamide Paracetamol or acetaminophen is a widely used over-the-counter analgesic (pain reliever) and antipyretic (fever reducer). It is commonly used non-steroidal analgesic agent for the relief of fever, headaches, and other minor aches and pains, and is a major ingredient in numerous cold and flu remedies.

While acetaminophen has fewer gastro-intestinal side effects than aspirin, another commonly used non-steroidal analgesic agent, acute and chronic acetaminophen toxicity can result in gastro-intestinal symptoms, severe liver damage, and even death. The precise intermediates in the acetaminophen toxic metabolite pathway are not yet known. As indicated herein before, it had been thought that when acetaminophen was ingested, the cytochrome P-450 dependent enzyme system of the liver produced a potentially toxic metabolite of acetaminophen which was the cause of acetaminophen toxicity.

It was further believed that when safe amounts of acetaminophen had been ingested, this toxic metabolite was cleared by hepatic glutathione stores. However in the case of acute or chronic overdose, excessive levels of the toxic metabolite were thought to delete the glutathione stores in the liver, resulting in hepatic necrosis. Later studies have proposed that acetaminophen induced hepatic necrosis may be due to cellular oxidative stress, resulting both in lipid peroxidation, protein and non-protein thiol oxidation, and changes in the intracellular calcium homeostasis. Symptoms of acute acetaminophen toxicity are typically mild or non-existent until at least 48 hours post-ingestion.

Thus, in yet another embodiment the acute or chronic toxic effect of acetaminophen treated by the combined composition of the invention may be any one of drug induced liver injury (DILI), drug-induced acute steatosis, cytotoxic hepatocellular injury, acute liver failure (ALF), reperfusion injury, ischemic liver disease and acute cholestatic injury.

According to one specific embodiment, the pharmaceutical combined composition of the invention is particularly applicable for treating, preventing, ameliorating, reducing or delaying the onset of drug induced liver injury (DILI), caused by acetaminophen.

It should be appreciated that the different Cytochrome P-450 inducing or inhibiting drugs may lead to different hepatic injuries, and therefore, may be prevented or treated by the combined compositions of the invention. For example, chlorpromazine, phenylbutazone, halogenated anesthetic agents and sulindac may cause fever, rash and eosinophilia. Dapsone may lead to sulfone syndrome (i.e., fever, rash, anemia, and jaundice), INH (Isoniazid (Laniazid, Nydrazid), also known as isonicotinylhydrazine (INH) and halothane may cause acute viral and or bacterial and or fungal and or parasitic hepatitis, Chlorpromazine, erythromycin, amoxicillin- and clavulanic acid may lead to obstructive jaundice. Phenytoin, carbamazepine, Phenobarbital and primidone may cause anticonvulsant hypersensitivity syndrome (i.e., triad of fever, rash, and liver injury), Para-amino salicylate, phenytoin, sulfonamides, may lead to serum sickness syndrome, Clofibrate may lead to Muscular syndrome (i.e., myalgia, stiffness, weakness, elevated creatine kinase level), Procainamide may cause Antinuclear antibodies (ANAs), Gold salts, propylthiouracil, chlorpromazine and chloramphenicol may cause marrow injury. Drugs such as Amiodarone and nitrofurantoin may be lead to associated pulmonary injury and Gold salts, methoxyflurane, penicillamine, paraquat may also lead to Associated renal injury. Tetracycline may cause Fatty liver of pregnancy, Contraceptive and anabolic steroids and rifampin may cause bland jaundice, Aspirin may cause Reye syndrome, and Sodium valproate may lead to Reye like syndrome.

Still further, other acute hepatocellular injuries caused by drugs may be treated or prevented by the combined compositions of the invention. For example, acute viral and or bacterial and or fungal and or parasitic hepatitis-like picture may be caused by INH, halothane, diclofenac and troglitazone. Mononucleosis like picture may be a result of using phenytoin, sulfonamides or dapsone. Chronic hepatocellular injury may be a result of Pemoline or methyldopa. Massive necrosis may be a result of using acetaminophen, halothane or diclofenac.

Steatosis may also be a result of using different drugs, for example, Macro vesicular steatosis may be caused by Alcohol, methotrexate, corticosteroids, minocycline, nifedipine and TPN, Microvesicular steatosis may be caused by alcohol, valproic acid, tetracycline and piroxicam. Steatohepatitis may be a result of Amiodarone, nifedipine, synthetic estrogens and didanosine. Pseudoalcoholic injury may be caused by Amiodarone, Acute cholestasis maybe a result of using Amoxicillin-clavulanic acid, erythromycin and sulindac. Chronic cholestasis may be caused by Chlorpromazine, sulfamethoxazole-trimethoprim, tetracycline or ibuprofen. Granulomatous hepatitis may be a result of using Carbamazepine, allopurinol and hydralazine. Vascular injury may be caused by steroids, Neoplasia may be a result of using Contraceptives or anabolic steroids. Adenoma may be caused by steroids, Angiosarcoma may be a result of Vinyl chloride. Hepatocellular carcinoma may be caused by Anabolic steroids, aflatoxin, arsenic or vinyl chloride.

More particularly, a drug such as Amoxicillin may cause hepatic dysfunction including jaundice, hepatic cholestasis, and acute cytolytic hepatitis.

Statins are among the most widely prescribed medications in the western world. The use of statins/HMG-CoA reductase inhibitors is associated with biochemical abnormalities of liver function, and thus may be also prevented or treated by the combined composition of the invention. Moderate elevations of serum transaminase levels (<3 times the upper limit of the reference range) have been reported following initiation of therapy and are often transient. Elevations are not accompanied by any symptoms and do not require interruption of treatment. Persistent increases in serum transaminase levels (>3 times the upper limit of the reference range) occur in approximately 1% of patients, and these patients should be monitored until liver function returns to normal after drug withdrawal. Active liver disease or unexplained transaminase elevations are contraindications to use of these drugs. Patients with a recent history of liver disease or persons, who regularly consume alcohol in large quantities, should use statins in a regulated manner.

In certain embodiments, the combined compositions of the invention may also be applicable for preventing and treating liver injury caused by Rifampin. Rifampin is usually administered with INH. On its own, rifampin may cause mild hepatitis, but this is usually in the context of a general hypersensitivity reaction. Fatalities associated with jaundice have occurred in patients with liver disease and in patients taking rifampin with other hepatotoxic agents. Careful monitoring of liver function (especially SGPT/SGOT) should be performed prior to therapy and then every 2-4 weeks during therapy. In some cases, hyper-bilirubinemia resulting from competition between rifampin and bilirubin for excretory pathways of the liver can occur in the early days of treatment. Isolated cholestasis also may occur.

In yet a further embodiment, the combined compositions of the invention may be applicable for preventing or treating liver damage caused by Valproic acid and divalproex sodium. More specifically, microvesicular steatosis is observed with alcohol, aspirin, valproic acid, amiodarone, piroxicam, stavudine, didanosine, nevirapine, and high doses of tetracycline. Prolonged therapy with methotrexate, INH, ticrynafen, perhexiline, enalapril, and valproic acid may lead to cirrhosis. Valproic acid typically causes microsteatosis. This drug should not be administered to patients with hepatic disease and may be used with caution in patients with a prior history of hepatic disease. Those at particular risk include children younger than 2 years, those with congenital metabolic disorders or organic brain disease, and those with seizure disorders treated with multiple anticonvulsants.

Hepatic failures resulting in fatalities have occurred in patients receiving valproic acid. These incidents usually occur during the first six months of treatment and are preceded by nonspecific symptoms such as malaise, weakness, lethargy, facial edema, anorexia, vomiting, and even loss of seizure control.

It should be further appreciated that the combined compositions of the invention may also be used for preventing or treating liver damage caused by using herbs. The increasing use of alternative medicines has led to many reports of toxicity. The spectrum of liver disease is wide with these medicines, for example: Senecio/crotalaria (Bush teas) can cause venoocclusive disease. Germander in teas is used for its anticholinergic and antiseptic properties. Jaundice with high transaminase levels may occur after two months of use, but it disappears after stopping the drug. Chaparral is used for a variety of conditions, including weight loss, cancer, and skin conditions. It may cause jaundice and fulminant hepatic failure. Chinese herbs have also been associated with hepatotoxicity.

According to certain embodiments, the compositions and combined compositions of the invention may also be applicable in treating liver damage caused by recreational drugs. More specifically, Ecstasy is an amphetamine used as a stimulant and may cause hepatitis and cirrhosis. Cocaine abuse has been associated with acute elevation of hepatic enzymes. Liver histology shows necrosis and microvascular changes.

More specifically, according to some embodiments, in addition to the enhancement or the augmentation of the beneficial effect of insulin whether via a direct or an indirect adjuvant effect, as described above, the pharmaceutical composition of the invention is intended for treating, preventing, ameliorating, reducing or delaying the onset of acute or chronic toxic effect of insulin.

In specific embodiments, said compositions are applicable to counteract toxic effects of analgesic or antipyretic drugs given in a separate formulation without jeopardizing their beneficial therapeutic effects. More specifically, such compositions may be administered concomitantly with at least one additional therapeutic agent selected from analgesic or antipyretic drug. Such analgesic or antipyretic drug may be according to certain embodiments, an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate. According to one specific embodiment, the invention relates to a combined composition comprising SE, C: E and acetaminophen, thereby providing a safe preparation of acetaminophen, having reduced potential for hepatic toxicity.

According to certain specific embodiments, the composition and combined compositions of the invention is particularly suitable for oral or mucosal administration. The usefulness of an oral formulation requires that the active agent or combinations of the invention be bioavailable. Bioavailability of orally administered drugs can be affected by a number of factors, such as drug absorption throughout the gastrointestinal tract, stability of the drug in the gastrointestinal tract, and the first pass effect. Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the composition of the invention may be suitable for mucosal administration, for example, pulmonary, buccal, nasal, intranasal, sublingual, rectal, dermal, vaginal administration and any combination thereof.

Although preferred administration is oral or mucosal, it should be appreciated that the composition of the invention may be also suitable for intravenous, intramuscular, subcutaneous, intraperitoneal, perenteral, transdermal, sublingual, topical, administration, or any combination thereof.

In another aspect, the invention further relates to an oral or mucosal pharmaceutical composition made by combining a therapeutically effective amount of at least one natural or synthetic soy extracts or any fractions thereof and at least one polyethoxylated castor oil or any derivative thereof, and optionally at least one additional therapeutic agent, with a pharmaceutically acceptable carrier.

In some embodiments, the oral or mucosal composition of the invention may comprise as an active ingredient soy fractions and at least one adjuvant, for example, PEG and/or beta cyclo dextrin, or any combination thereof.

According to a specifically preferred embodiment, such composition is as described by the invention. Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a naso gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

In yet another of its aspects, the present invention provides a method for controlling blood sugar levels in a subject, treating liver damage, restoring liver function and for treating, preventing, ameliorating, treating an immune related disorder, reducing or delaying the onset of acute or chronic toxic effect of alcohol or of a drug, said method comprises providing to a subject at least one of: (a) at least one soy extract or any fraction thereof; (b) at least one polyethoxylated castor oil or any derivative thereof and/or optionally, at least one adjuvant selected from polyethylene glycol and beta cyclo dextrin or any derivative thereof; (c) any combination of (a) and (b); and (d) a composition comprising any one of (a), (b) or (c).

In this connection, it should be understood that the terms treatment or prevention as used herein refers to the complete range of therapeutically positive effects of administrating to a subject including inhibition, reduction of, alleviation of, and relief from, a condition, illness, symptoms or undesired side effects thereof. These also include treatment or prevention of recurrence of a disease in response to a treatment with a non-effective, or deleterious therapeutic agent, and prevention or postponement of disease development, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms inhibition, moderation, reduction or attenuation as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with fold change values, i.e., 0.1, 0.5, 1.2, 5, etc., respectively.

The term prevention is interchangeable with prophylaxis in referring to significant reduction of risk of occurrence of a biological or medical event that is sought to be prevented in a tissue, a system, animal or human by a researcher, veterinarian, medical doctor or other clinician, and the term prophylactically effective amount is intended to mean that amount of a pharmaceutical composition that will achieve this goal.

For the purpose of specific methods according to the above a subject is provided at least one SE or any fraction thereof and optionally at least one polyethoxylated castor oil or any derivative thereof or any combination thereof.

In yet other embodiments, methods according to the invention may include administering or providing at least one soy derived polar or non polar fraction and/or at least one polyethoxylated castor oil or any derivative thereof, and/or any combination thereof and/or a composition comprising the same

Those methods using soy derived polar fraction, in certain embodiments thereof, may comprise administering of at least one of phospholipids, phosphatides or a combination thereof, which can be natural or synthetic.

In specific embodiments, the methods using phosphatides may comprise administering of any one of natural or synthetic phosphatidylcholine (PC), phosphatidylinositol (PI) or a combination thereof, which are particularly characteristic of the polar fraction presently designated as M1.

Those methods using soy derived non-polar fraction, in certain embodiments, may comprise administering of at least one of glycerides, phospholipids and phosphatides, which can be natural or synthetic.

In specific embodiments, the methods using glycerides, phospholipids and phosphatides may comprise administering of any one of phosphatidic acid (PA), phosphatidylethanolamine (PE) and phosphatidylcholine (PC), which are characteristic of the non-polar fraction presently designated as OS.

Further, in specific embodiments, methods using a polyethoxylated castor oil derivative, alone or in combination with SE or SE fraction, may use a commercially available derivative of synthetic polyethoxylated castor oil, Cremophore EL (C:E).

Thus, it is conceived that methods using any of the above compositions of the invention are applicable for controlling blood sugar levels in a subject, treating an immune related disorder, treating liver damage, restoring liver function and for treating, preventing, ameliorating, reducing or delaying the onset of acute or chronic toxic effect of a drug on any body organs or tissues. Detailed discussion on clinical conditions that are relevant to the present invention is presented further below.

Particularly for pre-clinical applications, methods of the invention use any of the above compositions in formulations adapted for add-on to a solid, semi-solid or liquid food, beverage, food additive, food supplement, medical food, botanical drug, drug and/or a pharmaceutical compound.

In specific pre-clinical applications, methods of the invention use the above compositions as add-on to foods and/or beverages comprising an increased content of sugar and/or alcohol.

Methods using the above compositions of the invention are particularly applicable for controlling blood sugar levels in a subject, wherein said control is inhibiting increase or decrease in blood sugar levels, improving glucose tolerance or altering insulin resistance state.

In certain embodiments, methods of the present invention are applicable to the prevention or alleviation of symptoms related to a condition associated with increased or decreased blood sugar levels, wherein said condition is any one of a hepatic disorder, pancreatic dysfunction, diabetes, obesity, weight gain, alcohol intoxication, alcohol withdrawal, vertigo, and tissue or organ damage or any condition associated with alteration of pancreatic or liver function in a way that alter insulin resistance and liver metabolic capability.

The present invention is directed at treating, controlling or preventing a number of medical conditions. In general, the terms preventing, controlling and treating encompass a range of conditions, starting from prevention of the development of a disease or a symptom in a patient who may predisposed to a disease but has yet been diagnosed; further including reduction, retardation or inhibition of progression symptoms of a disease; and also alleviation of symptoms of an already existing disease, i.e. reversal of said symptoms.

Methods and compositions of the invention are specifically relevant to treating, controlling, ameliorating, or preventing body weight gain, obesity, metabolic syndrome and diabetes.

By body weight gain is meant specifically body fat gain that is maintained or decreased by applying the methods and compositions of the invention. A decrease in weight or body fat may protect against cardiovascular disease by lowering blood pressure, total cholesterol, LDL cholesterol and triglycerides, and may alleviate symptoms associated with chronic conditions such as hypertension, coronary heart disease, type 2 diabetes, osteoarthritis, sleep apnea and degenerative joint disease.

The present invention is applicable to all types of obesity, including endogenous obesity, exogenous obesity, hyper-insulinar obesity, hyperplastic-hypertrophic obesity, hypertrophic obesity, hypothyroid obesity and morbid obesity. Moreover, inflammation-mediated obesity may be treated particularly effectively in accordance with the invention.

By metabolic syndrome, or syndrome X, is meant a complex multi-factorial condition accompanied by an assortment of abnormalities including hypertension, hyper-triglyceridemia, hyperglycemia, low high-density lipoprotein (HDL) cholesterol and abdominal obesity, which, among others, may lead to pro-thrombotic (e.g., elevated fibrinogen or plasminogen activator inhibitor-1 in the blood) and pro-inflammatory (e.g., elevated C-reactive protein (CRP) in the blood) conditions.

The World Health Organization (WHO) guidelines for diagnosis of metabolic syndrome are (Journal of Hypertension, Volume 17, pages 151-183, 1999):
hypertension (>140 mm Hg systolic or >90 mm Hg diastolic).
dyslipidemia, defined as elevated plasma triglycerides (150 mg/dL), and/or low high-density lipoprotein (HDL) cholesterol (<35 mg/dL in men, <39 mg/dL in women).
visceral obesity defined as a high body mass index (BMI) (30 kg/m2) and/or a high waist-to-hip ratio (>0.90 in men, >0.85 in women).
microalbuminuria (urinary albumin excretion rate of 20 g/min).

Alternatively, according to the National Cholesterol Education Program (NCEP) metabolic syndrome if at least three of the following five symptoms are present (JAMA, Volume 285, pages 2486-2497, 2001):
waist circumference >102 cm (40 in) for men or >88 cm (37 in) for women.
triglyceride level of 150 mg/dL.
HDL cholesterol level <40 mg/dL for men or <50 mg/dL for women.
blood pressure >130/85 mm Hg.
fasting glucose >110 mg/dL.

Each of the disorders associated with metabolic syndrome are risk factors in their own right, and can promote atherosclerosis, cardiovascular disease, stroke, and other adverse health consequences. However, when present together, these factors are predictive of increased risk of cardiovascular disease and stroke.

In the context of the present invention, controlling or treating metabolic syndrome using the combined compositions of the invention, is meant reducing severity and/or number of symptoms associated with this medical condition, i.e. reducing any one of elevated blood glucose, glucose intolerance, insulin resistance, elevated triglycerides, elevated LDL-cholesterol, low HDL cholesterol, elevated blood pressure, abdominal obesity, pro-inflammatory states, and pro-thrombotic states. Additionally or alternatively, it is meant reducing the risk and/or the onset of developing associated diseases, i.e. cardiovascular disease, coronary heart disease and other diseases related to plaguing of the artery walls and diabetic conditions.

Further, methods and compositions of the invention are particularly advantageous for treating, controlling and preventing diabetes or diabetic conditions, such as type 1 diabetes, type 2 diabetes, gestational diabetes, pre-diabetes, slow onset autoimmune diabetes type 1 (LADA), hyperglycemia or any type of condition or compound that expose the patient to pre diabetes or to diabetes or that alters the stage of insulin resistance. For the purposes of treatment, the diabetes may be overt, diagnosed diabetes, e.g., type 2 diabetes, or a pre-diabetic condition.

Diabetes mellitus (generally referred to herein as diabetes) is a disease that is characterized by impaired glucose regulation. Diabetes is a chronic disease that occurs when the pancreas fails to produce enough insulin or when the body cannot effectively use the insulin that is produced, resulting in an increased concentration of glucose in the blood (hyperglycemia). The WHO recognizes three main forms of diabetes mellitus: type 1, type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues, this causes a need for abnormally high amounts of insulin and diabetes develops when the beta cells cannot meet this demand. Gestational diabetes is similar to type 2 diabetes in that it involves insulin resistance, hormones in pregnancy may cause insulin resistance in women genetically predisposed to developing this condition.

Type 1 diabetes is also recognized as insulin-dependent, juvenile, or childhood-onset diabetes; type 2 diabetes—as non-insulin-dependent or adult-onset diabetes; LADA diabetes is late autoimmune diabetes of adulthood. Additionally, intermediate conditions such as impaired glucose tolerance and impaired fasting glycemia are recognized as conditions that indicate a high risk of progressing to type 2 diabetes.

In type 1 diabetes, insulin production is absent due to autoimmune destruction of pancreatic beta-cells. There are several markers of this autoimmune destruction, detectable in body fluids and tissues, including islet cell autoantibodies, insulin autoantibodies, glutamic acid decarboxylase autoantibodies, and tyrosine phosphatase ICA512/IA-2 autoantibodies. In type 2 diabetes, comprising 90% of diabetics worldwide, insulin secretion may be inadequate, but peripheral insulin resistance is believed to be the primary defect. Type 2 diabetes is commonly, although not always, associated with obesity, a cause of insulin resistance. It should be further appreciated that the method of the invention is applicable for a subject displaying increased insulin resistance.

Type 2 diabetes is often preceded by pre-diabetes, in which blood glucose levels are higher than normal but not yet high enough to be diagnosed as diabetes. The term pre-diabetes, as used herein, is interchangeable with the terms impaired glucose tolerance or impaired fasting glucose, which are terms that refer to tests used to measure blood glucose levels.

Chronic hyperglycemia in diabetes is associated with multiple, primarily vascular complications affecting microvasculature and/or macrovasculature. These long-term complications include retinopathy (leading to focal blurring, retinal detachment, and partial or total loss of vision), nephropathy (leading to renal failure), neuropathy (leading to pain, numbness, and loss of sensation in limbs, and potentially resulting in foot ulceration and/or amputation), cardiomyopathy (leading to heart failure), and increased risk of infection. Type 2, or noninsulin-dependent diabetes mellitus (NIDDM), is associated with resistance of glucose-utilizing tissues like adipose tissue, muscle, and liver, to the physiological actions of insulin. Chronically elevated blood glucose associated with NIDDM can lead to debilitating complications including nephropathy, often necessitating dialysis or renal transplant; peripheral neuropathy; retinopathy leading to blindness; ulceration and necrosis of the lower limbs, leading to amputation; fatty liver disease, which may progress to cirrhosis; and susceptibility to coronary artery disease and myocardial infarction. By ‘prevent’ it is meant that the risk of developing of diabetes is reduced or the onset of the disease is delayed. By ‘control’ or ‘treat’ it is meant that the risk of developing associated complications is reduced and/or the onset of such complications is delayed.

Diabetic conditions that are subject to treatment with SE or CE or their combinations or their combinations with other drugs, and with insulin, according to the methods of the present invention can be diagnosed or monitored using any of a number of assays known in the field. Examples of assays for diagnosing or categorizing an individual as diabetic or pre-diabetic or monitoring said individual include, but are not limited to, a glycosylated hemoglobin (HbA1c) test, a connecting peptide (C-peptide) test, a fasting plasma glucose (FPG) test, an oral glucose tolerance test (OGTT), and a casual plasma glucose test.

HbA1c is a biomarker that measures the amount of glycosylated hemoglobin in the blood. HbA1c designates a stable minor glycated sub fraction of hemoglobin. It is a reflection of the mean blood glucose levels during the last 6-8 weeks, and is expressed in percent (%) of total hemoglobin. Alternatively, diabetes or pre-diabetes can be diagnosed by measuring blood glucose levels using any of several known tests in the field, including a fasting plasma glucose test or an oral glucose tolerance test. Using the fasting plasma glucose (FPG) test, a patient is classified as diabetic and is subject to treatment according to the methods of the present invention if the patient has a threshold FPG greater than 125 mg/dl, and a patient is classified as pre-diabetic and is subject to treatment according to the methods of the present invention if the patient has a threshold FPG greater than 100 mg/dl but less than or equal to 125 mg/dl. Using the oral glucose tolerance test (OGTT), a patient is classified as diabetic and is subject to treatment according to the methods of the present invention if the patient has a threshold 2-hour OGTT glucose level greater than 200 mg/dl. A patient is classified as pre-diabetic and is subject to treatment according to the methods of the present invention if the patient has a threshold 2-hour OGTT glucose level greater than 140 mg/dl but less than 200 mg/dl.

C-peptide, produced from proinsulin molecules, is secreted from islet cells into the bloodstream in equimolar proportion as insulin, and is used a biomarker for beta-cell function and insulin secretion. A fasting C-peptide measurement greater than 2.0 ng/ml is indicative of high levels of insulin, while a fasting C-peptide measurement less than 0.5 ng/ml indicates insufficient insulin production.

According to another embodiment, methods according to the invention may further lead to a significant reduction in pancreatic hyperplasia and hepatic fat accumulation.

Still further, according to another embodiment, methods according to the invention may downregulate the function of macrophages while increasing foxp3+ or any other type of regulatory T cells in fat tissue or in the body, suppresses inflammatory cytokine production by adipocytes and clearly leads to a marked decrease of inflammatory cell infiltration to fat tissue of a treated subject, specifically, a subject suffering from an immune-related disorder.

More particularly, methods of the invention are intended for treatment of dyslipoproteinemia, which may include hypertriglyceridemia, hypercholesterolemia and low HDL-cholesterol, obesity, NIDDM (non-insulin dependent diabetes mellitus), IGT (impaired glucose tolerance), blood coagulability, blood fibronolysis defects and hypertension.

According to certain embodiments, the immunomodulatory composition of the invention is especially advantageous for the treatment of type 1 diabetes or diabetes mellitus, thereby preventing or reducing acute complications (e.g. hypoglycemia, ketoacidosis or nonketotic hyperosmolar coma) as well as long-term complications (e.g. cardiovascular disease, chronic renal failure, retinal damage or blindness, nerve damage and microvascular damage, which may cause impotence, poor healing wounds particularly of the feet potentially leading to gangrene and amputation).

According to some embodiments of, methods and compositions of the invention can be used to prevent, treat and control liver diseases and disorders including hepatitis, cirrhosis, non-alcoholic steatohepatitis (NASH) (also known as non-alcoholic fatty liver disease-NAFLD), hepatotoxicity and chronic liver disease. In general, the terms ‘prevent’, ‘control’ and ‘treat’ encompass the prevention of the development of a disease or a symptom from a patient who may have a predisposition of the disease or the symptom but has yet been diagnosed to have the disease or the symptom; the inhibition of the symptoms of a disease, namely, inhibition or retardation of the progression thereof; and the alleviation of the symptoms of a disease, namely, regression of the disease or the symptoms, or inversion of the progression of the symptoms.

In further embodiments, such methods may optionally further comprises the concurrent or parallel administration of an additional therapeutic agent. In some specific embodiments, such additional therapeutic agent may be any one of insulin, N-acetyl cysteine (NAC), thiamine (vitamin B1), a benzodiazepine, any gut hormone as described above, or any combination thereof.

In yet other embodiments, said methods may be applied for treating a subject suffering from a disorder associated with increased or decreased blood sugar levels.

For specific applications of the invention, said disorder may any one of a hepatic disorder, pancreatic dysfunction, diabetes, obesity, insulin resistance, metabolic syndrome, alcohol intoxication, alcohol withdrawal, vertigo, and tissue or organ damage.

Specific applications of the invention include, treating, preventing, ameliorating, reducing or delaying the onset of an immune-related disorder. The method comprising the step of administering a therapeutically effective amount of at least one soy derived polar fraction and at least one polyethoxylated castor oil, any derivative thereof, any combination thereof, or any composition comprising the same.

In specific embodiments, said immune-related disorder is any one of an inflammatory disorder, an autoimmune disorder, an infectious disease and a proliferative disorder.

Immune therapy involves the exposure of components of the immune system to various elements (cytokines, disease associated antigens and natural metabolites) to combat disease processes in which a dysregulated immune response is thought to play a role Immune dysregulation is thought to play a major part in the pathogenesis or disease course of a great number of disease processes, including various neoplastic, inflammatory, autoimmune, infectious and genetic entities.

These disorders can be perceived as a dysbalance between pro-inflammatory (Th1) and anti-inflammatory (Th2) cytokines. Or any dysbalance of cells that control the immune system whether, being a regulatory cell of any kind, antigen presenting cells, or any cells capable of altering the immune system. The way the immune system responds to foreign and self antigens, is the result of a balance between the two subtypes of responses. Experimental autoimmune diseases in humans can be perceived as a dysbalance between pro-inflammatory and anti-inflammatory cytokines, or a dysbalance between cells or cytokines or chemokines.

In the past few years it has been become increasingly clear that T cells capable of actively suppressing immune responses are thought to be in part responsible for the maintenance of peripheral self tolerance. In healthy rodents and humans, there are different types of cells which are able to exert such suppressive function in vitro and in vivo Immunoregulatory cytokines such as IL-10 or TGF-β may be critical for the suppressive effect of these cells. Regulatory T cells have potential role in human autoimmune or chronic inflammatory diseases and can be used for diagnostic or therapeutic purposes.

In more specific embodiments, such immune-related disorder may be any one of an inflammatory disorder, an autoimmune disorder, an infectious disease and a proliferative disorder.

In yet other embodiments, methods of the invention may be used for the treatment of an autoimmune disorder. Examples of autoimmune disorders include, but are not limited to, Asthma, Primary sclerosing cholangitis, Alopecia Areata, Lupus, Anlcylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Vasculitis, Lichen Planus, and Vitiligo. The oral combined SE and Cremophore EL compositions described herein can be administered to a subject to treat or prevent disorders associated with an abnormal or unwanted immune response associated with cell, tissue or organ transplantation, e.g., renal, hepatic, and cardiac transplantation, e.g., graft versus host disease (GVHD), or to prevent allograft rejection.

According to specific embodiments, an autoimmune disease treated by methods of the invention may be any one of rheumatoid arthritis, type 1 diabetes, type 2 diabetes, atherosclerosis, asthma, acute and chronic graft versus host disease, systemic lupus erythematosus, scleroderma, multiple sclerosis, inflammatory bowel disease, psoriasis, uvietis, thyroiditis and immune mediated hepatitis.

According to other embodiments, methods of the invention are applicable to the treatment of Multiple Sclerosis (MS). MS is typically characterized clinically by recurrent or chronically progressive necrologic dysfunction, caused by lesions in the CNS. Pathologically, the lesions include multiple areas of demyelination affecting the brain, optic nerves, and spinal cord. The underlying etiology is uncertain, but MS is widely believed to be at least partly an autoimmune or immune-mediated disease.

Thus, the invention includes compositions and methods for treating, delaying or preventing the onset of MS, by administering the combined SE and C:E. Included are methods wherein a subject who has or is at risk of having MS is administered combined SE and C:E.

According to another preferred embodiment, methods of the invention may be used for treating any inflammatory arthritis. In specific embodiments, the compositions and methods of the invention may be applicable for treating Rheumatoid arthritis (RA). Rheumatoid arthritis (RA) is the most common chronic inflammatory arthritis and affects about 1% of adults, it is two to three times more prevalent in women than in men. RA may begin as early as infancy, but onset typically occurs in the fifth or sixth decade.

Diagnosis may be made according to the American Rheumatism Association Criteria for the so Classification of Rheumatoid Arthritis. A therapeutically effective amount will cause an improvement in one or more of the following: the number of inflamed joints, the extent of swelling, and the range of joint motion. Laboratory measurements (e.g., ESR and hematocrit value) and assessments of subjective features (e.g., pain and morning stiffness) can also be made. The invention also includes methods of treating autoimmune arthritis, e.g., RA, in a subject by administering to the subject a therapeutically effective amount of combined composition of the invention comprising SE and Cremophore EL.

Methods of the invention described herein can also be used to treat or prevent graft rejection in a transplant recipient. For example, methods of the invention can be used in a wide variety of tissue and organ transplant procedures, e.g., can be used to induce central tolerance in a recipient of a graft of cells, in stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail cell, tissue or organ transplantation (e.g. liver transplantation to treat hypercholesterolemia, transplantation of muscle cells to treat muscular dystrophy, or transplantation of neuronal tissue to treat Huntington's disease or Parkinson's disease).

According to another embodiment, methods of the invention may modulate the T cells or other cells balance towards a suppressing response in a subject suffering from IBD. Therefore, according to one embodiment, the composition of the invention is intended for treating IBD. Inflammatory bowel diseases (IBD) are common gastrointestinal disorders that can be perceived as being the result of a dysbalance between pro-inflammatory and anti-inflammatory subtypes of immune responses.

Patients with IBD have antibodies against components of colon cells and several different bacterial antigens. These antigens gain access to the immune system as a consequence of epithelial damage. Abnormalities of T cell-mediated immunity, including coetaneous anergy and diminished responsiveness to T cell stimuli, have also been described in these patients. In addition, changes in mucosal cell mediated immunity were identified, including increased concentrations of mucosal IgG cells and changes in T cells subsets, suggesting antigen stimulation. Exposure of target antigens after infectious, immune, or toxic damage, leads to activation of mucosal immune cells resulting in cytokines that lead to mucosal inflammatory response. Secretion of pro-inflammatory cytokines such as IFNγ, contributes to an increase in mucosal permeability, and has been described in animal models of IBD.

In yet other embodiments, methods and compositions of the invention may be used for the treatment of atherosclerosis. Atherosclerosis is a slowly progressive disease characterized by the accumulation of cholesterol within the arterial wall. The atherosclerotic process begins when LDL-C becomes trapped within the vascular wall. Oxidation of the LDL-C results in the bonding of monocytes to the endothelial cells lining the vessel wall. These monocytes are activated and migrate into the endothelial space where they are transformed into macrophages, leading to further oxidation of LDL-C. The oxidized LDL-C is taken up through the scavenger receptor on the macrophage leading the formation of foam cells. A fibrous cap is generated through the proliferation and migration of arterial smooth muscle cells, thus creating an atherosclerotic plaque. Lipids depositing in atherosclerotic legions are derived primarily from plasma apo B containing lipoproteins. These include chylomicrons, LDL-C, IDL, and VLDL. This accumulation forms bulky plaques that inhibit the flow of blood until a clot eventually forms, obstructing an artery and causing a heart attack or stroke.

Thus, in other specific embodiments, methods and compositions of the invention are intended for the treatment of a malignancy. In cancerous situations, modulation of the T cell balance may be in the direction of inducing a pro-inflammatory response or in augmenting the anti-tumor associated antigens immunity. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the compositions of the present invention may be used in the treatment of non-solid and solid tumors.

Malignancy, as contemplated in the present invention may be selected from the group consisting of carcinomas, melanomas, lymphomas, myeloma, leukemia and sarcomas. Malignancies that may find utility in the present invention can comprise but are not limited to hematological malignancies (including leukemia, lymphoma and myeloproliferative disorders), hypoplastic and aplastic anemia (both viral and or bacterial and or fungal and or parasitically induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepatocellular carcinoma, colon cancer, melanoma, myeloma, acute or chronic leukemia.

It should be noted that the immuno-modulatory methods and compositions of the invention may be applicable for treating infectious diseases caused by bacterial infections, viral and or bacterial and or fungal and or parasitic infections, fungal infections, or parasitic infections. More specifically, the viral and or bacterial and or fungal and or parasitic infection may be caused by any one of HBV, HCV or HIV.

In some specific embodiments, methods of the invention may be suitable for treating an immune-related disorder, for example, hepatitis.

Still further embodiments relate to methods for treating liver damage in a subject in need thereof. More specifically, such method uses a composition comprises a therapeutically effective amount of a natural or synthetic SE and polyethoxylated castor oil or any derivative thereof, or any combination thereof.

In more specific embodiments, such subject may be a subject suffering from a liver disease, that may be any one of viral and or bacterial and or fungal and or parasitic, alcoholic or autoimmune hepatitis, alcoholic or autoimmune cirrhosis, alcoholic fatty liver disease, non alcoholic fatty liver disease (NAFLD), any type of liver steatosis, for example, due to other disease such as Wilson's disease or alpha 1 anti trypsin deficiency, alcoholic or nonalcoholic steatohepatits (NASH), hepatocellular carcinoma, drug-induced liver disease and pediatric liver disease and any type of metabolic liver disease, for example, glycogen storage disease.

The terms liver disease or liver damage as used herein apply to many diseases and disorders that cause the liver to function improperly or to cease functioning, and this loss of liver function is indicative of liver disease. Thus, liver function tests are frequently used to diagnose liver disease. Examples of such tests include, but are not limited to, the following:
Assays to determine the levels of serum enzymes such as lactate dehydrogenase (LDH), alkaline phosphatase (ALP), aspartate aminotransferase (AST), and alanine aminotransferase (ALT), where an increase in enzyme levels indicates liver disease. One of skill in the art will reasonably understand that these enzyme assays indicate only that the liver has been damaged. They do not assess the liver's ability to function. Other tests can be used to assay a liver's ability to function.
Assays to determine serum bilirubin levels. Serum bilirubin levels are reported as total bilirubin and direct bilirubin. Normal values of total serum bilirubin are 0.1-1.0 mgdl (e.g., about 2-18 mmol/L). Normal values of direct bilirubin are 0.0-0.2 mg/dl (0-4 mmol/L). Increases in serum bilirubin are indicative of liver disease.
Assays to determine serum protein levels, for example, albumin and the globulins (e.g., alpha, beta, gamma). Normal values for total serum proteins are 6.0-8.0 g/dl (60-80 g/L). A decrease in serum albumin is indicative of liver disease. An increase in globulin is indicative of liver disease.

Other tests include prothrombin time, international normalized ratio, activated clotting time (ACT), partial thromboplastin time (PTT), prothrombin consumption time (PCT), fibrinogen, coagulation factors; alpha-fetoprotein, and alpha-fetoprotein-L3 (percent).

In some embodiments, methods of the invention may further comprise concurrent or parallel administration of at least one additional therapeutic agent.

In certain embodiments such agent is any one of insulin, antibodies directed to inflammatory cytokine, or antibodies such as anti TNF antibodies including humanized antibodies, statins, analgesics, chemotherapeutic agents and antibiotics.

In yet other embodiments, said additional therapeutic agent is any one of N-acetyl cysteine (NAC), thiamine (vitamin B1), a benzodiazepine or any combination thereof and a tissue derived preparation or compound.

In still further embodiment the additional therapeutic agent that may be an autologous protein-containing tissue extract, for example, colon or liver. Such extract comprises disease-associated antigens that modulate the immune response in the treated subject.

Further, methods of the invention, particularly those using compositions of the invention comprising a therapeutically effective amount of at least one SE or any fraction thereof and at least one polyethoxylated castor oil or any derivative thereof, or any combination thereof, are particularly applicable for treating liver damage and/or restoring liver function in a subject in need thereof.

More specifically, such methods are applicable for treating subjects suffering for example from a liver disease, said liver disease is any one of viral, bacterial, fungal or parasitic liver disease, alcoholic or autoimmune hepatitis, alcoholic or autoimmune cirrhosis, alcoholic fatty liver disease, non alcoholic fatty liver disease (NAFLD), liver steatosis, alcoholic or nonalcoholic steatohepatits (NASH), hepatocellular carcinoma, drug-induced liver disease and pediatric liver disease and any type metabolic liver disease, for example glycogen storage disease.

Specific embodiments of said methods are applicable for treating, preventing, ameliorating, reducing or delaying the onset of acute or chronic toxic effect of a drug and for restoring liver function.

Relying on the present examples of the invention, the combined composition of the invention has been shown as significantly ameliorating Con A induced hepatitis thereby establishing the feasibility of using the composition of the invention for treating any liver damage. One clinically important type of liver disease is hepatitis. Hepatitis is an inflammation of the liver that can be caused by viruses (e.g., hepatitis virus A, B and C (HAV, HBV, and HCV, respectively), chemicals, drugs, alcohol, inherited diseases, or the patient's own immune system (autoimmune hepatitis). This inflammation can be acute and resolve within a few weeks to months, or chronic, and persist over many years. Chronic hepatitis can persist for decades before causing significant symptoms, such as cirrhosis (scarring and loss of function), liver cancer, or death. Other important examples of the different diseases and disorders encompassed by the term “liver disease” and suitable for treatment or prevention or control using the compositions and methods of the present invention include, but are not limited to amebic liver abscess, biliary atresia, fibrosis, cirrhosis, coccidioidomycosis, delta agent, hepatocellular carcinoma (HCC), alcoholic liver disease, primary biliary cirrhosis, pyogenic liver abscess, Reye's syndrome, sclerosing cholangitis, and Wilson's disease. In some embodiments, the compositions and methods described herein are suitable for the treatment of liver disease characterized by the loss or damage of parenchymal liver cells. In some aspects, the etiology of this can be a local or systemic inflammatory response. As the ConA immune mediated hepatitis model, the beneficial effect of SE in this model forms the basis for its potential beneficial effect in any immune-related disease, in which the immune system plays a role in the pathogenesis thereof. Such immune-related diseases include infectious, inflammatory, and malignant disorders.

Liver failure occurs when large parts of the liver become damaged and the liver is no longer able to perform its normal physiological function. In some aspects, liver failure can be diagnosed using the above described assays of liver function or by a subject's symptoms. Symptoms that are associated with liver failure include, for example, one or more of the following, nausea, loss of appetite, fatigue, diarrhea, jaundice, abnormal/excessive bleeding (e.g., coagulopathy), swollen abdomen, mental disorientation or confusion (e.g., hepatic encephalopathy), sleepiness, and coma.

Chronic liver failure occurs over months to years and is most commonly caused by viruses (e.g., HBV and HCV), long-term/excessive alcohol consumption, cirrhosis, hemochromatosis, and malnutrition. Acute liver failure is the appearance of severe complications after the first signs of liver disease (e.g., jaundice) and includes a number of conditions, all of which involve severe hepatocyte injury or necrosis. In some embodiments, the compositions and methods described herein are particularly suitable for the treatment of hyperacute, acute, and subacute liver failure, fulminant hepatic failure and late onset fulminant hepatic failure, all of which are referred to herein as “acute liver failure.” Common causes for acute liver failure include, for example, viral and or bacterial and or fungal and or parasitic hepatitis, exposure to certain drugs and toxins (e.g., fluorinated hydrocarbons (e.g., trichloroethylene and tetrachloroethane), amanita phalloides (e.g., commonly found in the “death-cap mushroom”), acetaminophen (paracetamol), halothanes, sulfonamides, henytoins), cardiac-related hepatic ischemia (e.g., myocardial infarction, cardiac arrest, cardiomyopathy, and pulmonary embolism), renal failure, occlusion of hepatic venous outflow (e.g., Budd-Chiari syndrome), Wilson's disease, acute fatty liver of pregnancy, amebic abscesses, and disseminated tuberculosis.

The term hepatitis is used to describe a liver condition which implies injury to the liver characterized by the presence of inflammatory cells in the tissue of the organ. The condition can be self-limiting, healing on its own, or can progress to scarring of the liver. Hepatitis is acute when it lasts less than six months and chronic when it persists longer than six months. A group of viruses known as the hepatitis viruses cause most cases of liver damage worldwide. Hepatitis can also be due to toxins (notably alcohol), other infections or from autoimmune process. Hepatitis includes hepatitis from viral and or bacterial and or fungal and or parasitic infections, including Hepatitis A through E (A, B, C, D and E—more than 95% of viral and or bacterial and or fungal and or parasitic cause), Herpes simplex, Cytomegalovirus, Epstein-Barr virus, yellow fever virus, adenoviruses; non-viral and or bacterial and or fungal and or parasitic infections, including toxoplasma, Leptospira, Q fever, rocky mountain spotted fever, alcohol, toxins, including amanita toxin in mushrooms, carbon tetrachloride, asafetida, among others, drugs, including paracetamol, amoxycillin, antituberculosis medicines, minocycline and numerous others as described herein; ischemic hepatitis (circulatory insufficiency); pregnancy; autoimmune conditions, including Systemic Lupus Erythematosus (SLE); and non-alcoholic steatohepatitis.

Sterile inflammation is used to describe inflammation of the liver which is triggered by intracellular molecules released from dying cells that have lost integrity of their plasma membrane. This inflammation occurs in the absence of causative agents such as viruses or bacteria and alcohol. A number of intracellular molecules have been identified that can stimulate other cells to produce proinflammatory cytokines and chemokines. Such proinflammatory cellular molecules are thought to function by engaging receptors on cytokine-producing cells. If left untreated, sterile inflammation may progress to non-alcoholic fatty liver disease (NAFLD), non-alcoholic steatohepatitis (NASH) or cyrrhosis.

Non-alcoholic steatohepatitis or NASH is a condition of the liver in which inflammation is caused by a buildup of fat in the liver. NASH is part of a group of liver diseases, known as nonalcoholic fatty liver disease, in which fat builds up in the liver and sometimes causes liver damage that gets worse over time (progressive liver damage). “Non-alcoholic fatty liver disease” (NAFLD) is fatty inflammation of the liver which is not due to excessive alcohol use. It is related to insulin resistance and the metabolic syndrome, obesity, high cholesterol and triglycerides and diabetes, and may respond to treatments originally developed for other insulin resistant states (e.g. diabetes mellitus type 2), such as weight loss, metformin and thiazolidinediones. Non-alcoholic steatohepatitis (NASH) is the most extreme form of NAFLD, which is regarded as a major cause of cirrhosis of the liver of unknown cause.

Other factors that have been known to contribute to NASH include: surgery that shorten the intestines, the stomach, or both, such as jejunal bypass operation or biliopancreatic diversion; prolonged use of feeding tube or other method of receiving nutrition; certain drugs, including amiodarone, glucocorticoids, synthetic estrogens, and tamoxifen.

NASH is a condition that may get worse over time (called a progressive condition) and can cause scarring (fibrosis) of the liver, which leads to cirrhosis. “Cirrhosis” describes a condition in which liver cells have been replaced by scar tissue. The term “cirrhosis of the liver” or “cirrhosis” is used to describe a chronic liver disease characterized by replacement of liver tissue by fibrous scar tissue as well as regenerative nodules, leading to progressive loss of liver function. Cirrhosis is most commonly caused by fatty liver disease, including NASH, as well as alcoholism and hepatitis B and C, but also may be of unknown cause. Potentially life-threatening complications of cirrhosis are hepatic encephalopathy (confusion and coma) and bleeding from esophageal varices. Cirrhosis has historically been thought to be generally irreversible once it occurs, and historical treatment focused on preventing progression and complications. In advanced stages of cirrhosis, the only option is a liver transplant.

Each of the compounds above, specifically in the combined compositions and methods of the present invention can be used to treat, prevent or control chemical liver trauma and hepatotoxicity. Also chemical trauma or acute chemical trauma to the liver refers to serious injury which occurs to a patient over a short duration as a consequence of chemical toxicity, including drug-induced toxicity or trauma. Drug-induced acute liver trauma, including acetaminophen-induced acute liver trauma, is acute liver injury which occurs as a result or consequence of exposure to a drug (e.g., drug overdose), especially acetaminophen toxicity. Compounds according to the present invention are useful for reducing the injury to the liver which occurs from physical and chemical trauma, especially including drug-induced (drug overdose) and acetaminophen-induced acute liver trauma.

Hepatotoxocity is chemical liver trauma resulting from a hepatotoxic agent, or hepatotoxicity-inducing bioactive agent. The terms “hepatotoxic agent” and “a hepatotoxicity inducing bioactive agent” are used synonymously in context to describe compounds which often produce hepatotoxicity in patients administered such agents. Examples of hepatoxicity agents include, for example, anaesthetic agents, antiviral and or bacterial and or fungal and or parasitic agents, anti-retroviral and or bacterial and or fungal and or parasitic agents (nucleoside reverse transcriptase inhibitors and non-nucleoside reverse transcriptase inhibitors), especially anti-HIV agents, anticancer agents, organ transplant drugs (cyclosporin, tacrolimus, OKT3), antimicrobial agents (anti-TB, anti-fungal, antibiotics), anti-diabetes drugs, vitamin A derivatives, steroidal agents, especially including oral contraceptives, anabolic steroids, androgens, non-steroidal anti-inflammatory agents, anti-depressants (especially tricyclic antidepressants) glucocorticoids, natural products and herbal and alternative remedies, especially including St. John's wort.

Hepatotoxicity may manifest as triglyceride accumulation which leads to either small droplet (microvesicular) or large droplet (macrovesicular) fatty liver. There is a separate type of steatosis where phospholipid accumulation leads to a pattern similar to the diseases with inherited phospholipid metabolism defects (e.g. Tay-Sachs disease).

It must be understood that the combined compositions and methods of the invention are particularly applicable for treating any of the hepatic disorders described herein above.

In certain embodiments, the method of the invention may optionally further comprises the concurrent or parallel administration of at least one additional therapeutic agent. More specifically, such additional therapeutic agent may be any one of insulin, NAC, vitamin B1, a benzodiazepine, an anti-viral and or bacterial and or fungal and or parasitic or anti-inflammatory drug, a chemotherapeutic agent and any gut hormone. It is also conceived that for the purpose of specific embodiments, the methods, compositions and the combined compositions of the invention may be used as an add-on to any type of drugs or therapeutic compounds administered orally, intravenously, intradermaly, by inhalation or intrarectaly. Examples of such drugs or therapeutic compounds include, but are not limited to at least one of tissue derived antigens, tumor associated antigens, viral, bacterial, fungal, and parasitic derived antigens, as well as any type of organism derived antigens. The add-on composition of any type of SE with or without CE, or of any combination of different SE with or without CE according to the invention may be added to any type of tissue derived antigens obtained from a healthy or diseased subject, any type of drug or compound, any type of organism derived antigens, hormones, cytokines, therapeutic antibody, or any type of natural or non-natural therapeutic compound. The add-on composition of the invention may be used for promoting the effect of this compound, for exerting an adjuvant effect, or for improving the therapeutic effect of said therapeutic agent.

In further alternative embodiments, the method of the invention may be applicable for treating, preventing, ameliorating, reducing or delaying the onset of acute or chronic toxic effect of a drug.

In certain embodiments, such drug may be an analgesic or an antipyretic drug.

It is yet another important aspect of the present invention is to provide a pharmaceutical composition for use in a method for treating liver damage and/or restoring liver function in a subject in need thereof, said composition comprising as an active ingredient a therapeutically effective amount of at least one SE or any fraction thereof and least one a polyethoxylated castor oil or any derivative or a combination thereof, and optionally further comprising a pharmaceutically acceptable carrier. It should be appreciated that the composition of the invention may be used for treating, preventing and protecting from any damage caused by a therapeutic compound to any tissue or organ, and for restoring the biological function of said damaged tissue or organ.

A further aspect of the present invention is a soft, specifically high sugar or an alcoholic beverage comprising SE or any fraction thereof and optionally further comprising a polyethoxylated castor oil or any derivative or a combination thereof.

For certain purposes, such soft, specifically high sugar or alcoholic beverages comprising SE may comprise a soy derived polar or non-polar fraction.

In specific embodiments, said soft, specifically high sugar or alcoholic beverages may comprise specifically a polar fraction, or more specifically phosphatides characterizing this fraction or any one of phosphatidylcholine (PC), phosphatidylinositol (PI) or a combination thereof, which are characteristic of the polar fraction is designated M1.

In yet other specific embodiments, said soft, specifically high sugar or alcoholic beverages may comprise a non-polar fraction, or more specifically least one of glycerides, phospholipids and phosphatides), which characterize the non-polar fraction is designated OS.

Still further, the invention provides in some embodiments thereof at least one high sugar or alcoholic beverage, for example, SSB, chocolate milk and the like, that further comprise a combination of M1, OS, and optionally, at least one of C:E, cyclo dextrin and PEG or any derivative thereof.

The term ‘Sugar Sweetened Beverages’ (SSBs) as meant herein refers to beverages with high sugar content and/or those associated with higher caloric intakes. The 2010 Dietary Guidelines for Americans define SSBs as “liquids that are sweetened with various forms of sugars that add calories. These beverages include, but are not limited to, soda, fruit drinks, and sports and energy drinks”. In the National Health and Nutrition Examination Survey (NHANES) 2005-2008, about half of Americans drank SSBs on any given day. SSB intake in adults is associated with obesity, type 2 diabetes and increased risk for cardiovascular disease, nonalcoholic fatty liver disease, kidney disease, gout and decreased diet quality.

The present invention also relates to sweetened soft drinks (also soda, pop, soda pop, coke, soda pop, fizzy drink, seltzer, mineral, lolly water or carbonated beverage) that is a beverage that typically contains carbonated water, a sweetener and a flavoring. The sweetener may be sugar, high-fructose corn syrup, fruit juice, sugar substitutes (in the case of diet drinks) or some combination of these. An average can of sugared soda or juice contains about 10 to 12 teaspoons of sugar. Soft drinks may also contain caffeine, colorings, preservatives and other ingredients.

Among the popular SSBs, of particular relevance to the present context is Coca Cola (or coke), which for the purpose of the present invention refers to any carbonated soft drink flavored with coca leaves, cola nuts, caramel, etc, commercially available by other brand names.

Of further relevance to the present invention are milked beverages containing added sugars. Although not classified as SSBs, most flavored milks contain at least double the sugar of plain milk Flavored milk is cow's milk with added flavoring and sweetener, which is available in flavors such as chocolate, strawberry and vanilla flavors in low-fat and fat-free varieties. Most chocolate milks are sweetened with sugar or high fructose corn syrup.

In specific embodiments, the present invention applies to syrups and beverages containing sweeteners high in sugar (more than 95% sugar), such as brown sugar (97%), fructose (93%), honey (82%), high-fructose corn-syrup (76%), molasses (75%), agave syrup and maple syrup (68%), pancake syrups (42-68%), and Canadian maple syrup (60%).

In further embodiments, the invention applies to drink powders and drink concentrates high in sugar content (95% sugar), such as lemonade powder (95%), orange breakfast drink (92%), chocolate milk drink (84%), Gatorade mix (81%), melted chocolate drink mix (67%), cocoa mix powder (66%), instant coffee with whitener, reduced-sugar (59%), instant mocha coffee (58%), pink lemonade concentrate (46%), fruit drink (16%), cream soda and energy drink (13%), Cola, root beer and orange drink (11%), and lemon ice tea and lemon-lime soda (10%).

Yet in other embodiments, the present invention applies to foods with high sugar content, such as candies and nougat high in sugar (90% sugar), e.g. hard candies (93%), butterscotch (81%), vanilla fudge (80%), Skittles (76%), chocolate fudge (73%), chocolate coated fondant (71%), jelly beans and low calorie gum drops (70%), Taffy (69%), high vitamin C fruit snacks (68%), After Eight Mints (67%), chewing gum and caramels (66%).

The present invention further applies to foods containing dried fruits high in sugar (up to 80% sugar), such as blueberries, sweetened (68%), currants, dates and sweetened cherries (67%), cranberries, sweetened (65%), pears (62%), raisins (59%), apricots (53%), figs (48%), bananas (47%), peaches (42%), and prunes (38%).

In specific embodiments, the present invention applies to cookies, cakes and pies high in sugar (up to 70% sugar), such as chocolate sandwich cookies (61%), white cake with coconut frosting (57%), soft raisin cookies (48%), fortune cookies & chocolate covered marshmallows (45%), cream-filled wafers & coffee cake (43%), oatmeal cookies & yellow cake, with vanilla frosting (42%), chocolate cake (40%), diet chocolate chip cookies (40%), reduced fat chocolate brownies (39%), sugar cookies (38%), chocolate chip cookies & sponge cake (37%), coconut cream pie & Boston cream pie (36%), doughnuts, glazed (35%), blueberry muffins (33%), reduced-fat pie crust (30%), mince pies (28%), and pecan pie (25%).

The invention further applies to jams, preserves and spreads high in sugar (up to 60% sugar), such as chocolate-hazelnut spread (54%), most jams (49%), apricot jam (43%), diet jam (38%), chunky peanut butter (11%), and smooth peanut butter (10%).

In specific embodiments, the invention applies to cereals high in sugar (up to 56% sugar), such as Marshmallow Froot Loops (50%), Berry Colossal Crunch (44%), Cinnabon (42%), Frosted Rice Crispies (40%), Cocoa Crispies (39%), Frosted Flakes (38%), Cocoa Puffs (37%), Lucky Charms (36%), Golden Grahams (35%), Raisin Bran (34%), Low Fat Fruit Granola and Honey Nut Cheerios (33%), Special K Fruit And Yogurt (32%), Fruit And Nut Muesli (31%), Special K Red Berries (30%).

In specific embodiments, the invention applies to sauces and instant gravies high in sugar (up to 40%), such as cranberry sauce (38%), pickle relish (29%), Hoisin sauce (27%), pork gravy powder (25%), instant beef gravy (24%), peanut sauce (19%), sweet & sour sauce (19%), teriyaki sauce (14%), cocktail sauce (12%), tomato chili sauce (11%), pasta sauces (6-10%), cheese sauce mix, steak sauce and Worcestershire sauce (10%), instant turkey gravy (7%), salsa (4-6%), and Tartar sauce (4%).

In further embodiments, the invention applies to ice creams, frozen yogurts and milk shakes high in sugar (up to 25% sugar), such as chocolate ice cream and light chocolate ice cream (25%), frozen vanilla soft-serve yogurt (24%), light vanilla ice cream (22%), thick chocolate milk shake, vanilla ice cream and fat free vanilla ice cream (21%), 98% fat free chocolate ice cream (20%), chocolate frozen yogurt (19%), chocolate covered ice cream bar and thick vanilla milk shake (18%), non-fat, no sugar frozen yogurt (13%), fat-free, no sugar ice cream (9%).

Of particular relevance to the present invention are fruit canned in syrup high in sugar (up to 55% sugar), such as Maraschino cherries (39%), plums, sour red cherries and strawberries (22%), figs (21%), blueberries, raspberries, apricots & blackberries (20%), grapes & peaches (19%), fruit salad (18%), fruit cocktail & pineapple (17%), pears, sweet cherries (16%), and mandarin segments (15%).

The present invention is further relevant to alcoholic beverages. An alcoholic beverage is any fermented liquor, such as wine, beer, or distilled spirit, that contains ethyl alcohol, or ethanol (CH₃CH₂OH), as an intoxicating agent. In the US, a standard drink contains 0.6 ounces (14.0 grams or 1.2 tablespoons) of pure alcohol. Generally, this amount of pure alcohol is found in 12-ounces of beer (5% alcohol content); 8-ounces of malt liquor (7% alcohol content); 5-ounces of wine (12% alcohol content); 1.5-ounces of 80-proof (40% alcohol content) distilled spirits or liquor (e.g., gin, rum, vodka, whiskey).

Yet in another aspect, the present invention provides a combined composition comprising as an active ingredient at least one soy derived polar fraction and at least one polyethoxylated castor oil or any derivative thereof.

It is another aspect of the present invention to provide a pharmaceutical composition for use in a method for prevention of liver steatosis or liver disease in a healthy subject exposed to conditions inducing a liver disease, said composition comprising as an active ingredient a soy derived polar fraction and a polyethoxylated castor oil or any derivative or a combination thereof, and optionally further comprising a pharmaceutically acceptable carrier.

In yet another aspect, the present invention provides a pharmaceutical composition for use in a method for prevention of diabetes in a subject with pre diabetic condition, said composition comprising as an active ingredient a therapeutically effective amount of a SE or any fraction thereof and a polyethoxylated castor oil or any derivative or a combination thereof, and optionally further comprising a pharmaceutically acceptable carrier.

It is another important aspect of the present invention to provide a method for enhancing and augmenting the therapeutic effect of at least one therapeutic agent in a subject treated with said at least one of:

(a) at least one soy extract (SE) or any fraction thereof;

(b) at least one polyethoxylated castor oil and/or optionally, at least one adjuvant selected from polyethylene glycol and beta cyclo dextrin or any derivative thereof;

(c) any combination of (a) and (b); and

(d) a composition comprising any one of (a), (b) and (c).

Examples of such drugs or therapeutic compounds include, but are not limited to at least one of tissue derived antigens, tumor associated antigens, viral, bacterial, fungal, and parasitic derived antigens, as well as any type of organism derived antigens. Such therapeutic compound may be derived from any type of allogeneic, syngeneic or autologous tissue derived antigens obtained from a healthy or diseased subject, any type of drug or compound, any type of organism derived antigens, hormones, cytokines, therapeutic antibody, or any type of natural or non-natural therapeutic compound. The methods of the invention may be used for exerting an adjuvant effect and for promoting and improving the therapeutic effect of said therapeutic agent. It should be noted that in certain embodiments such augmenting and enhancing effect may be synergistic, additive, or adjuvant.

More specifically, according to some embodiments, in addition to the enhancement or the augmentation of the beneficial effect of a therapeutic compound or drug, e.g., insulin or any tissue or organ-derived antigen or preparation, whether via a direct or an indirect adjuvant effect, as described above, the pharmaceutical composition of the invention is intended for treating, preventing, ameliorating, reducing or delaying the onset of acute or chronic toxic effect of a therapeutic compound and drug.

It is understood that the methods of the invention involve administering the combined compositions of the invention, specifically, compositions comprising SE and polyethoxylated castor oil or any derivative or a combination thereof, particularly C:E. There are numerous administration routes that may be used. In some embodiments, the administration is at least one of oral, mucosal, nasal, transdermal, pulmonary, buccal or sublingual administration, or any combinations thereof. Other administration modes are also applicable, for example, subcutaneous, rectal, or parenteral (including intramuscular, intraperitoneal (IP), intravenous (IV) and intradermal) administration.

An amount adequate to accomplish this is defined as a “therapeutically effective dose.” Amounts effective for this use will depend upon the severity of the condition and the general state of the patient's own immune system, but generally range from about 0.001 to about 1000 mg/Kg of SE and/or C:E. Specifically, SE and C:E, in dosages of from 0.0001 to 5000 mg and 0.01 to 2.5, specifically, 0.001, 0.002, 0.003, 0.004, 0.005, 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 1.5, 2, 2.5, 3, 3.5, 4, 4.5 and 5 mg. More specifically, about 0.005 to 2.5 and most specifically, a low dose of 0.0.00001 mg or a high dose of 10000000 mg SE and C:E per Kg of body weight being more commonly used. Single or multiple administrations on a daily, weekly or monthly schedule can be carried out with dose levels and pattern being selected by the treating physician.

In yet another aspect, the present invention provides a composition for use in enhancing and augmenting the therapeutic effect of at least one therapeutic agent in a subject treated with said at least one therapeutic agent. More specifically, such composition may comprise as an active ingredient a therapeutically effective amount of at least one of:

(a) at least one soy extract (SE) or any fraction thereof;

(b) at least one polyethoxylated castor oil and/or optionally, at least one adjuvant selected from polyethylene glycol and beta cyclo dextrin or any derivative thereof;

(c) any combination of (a) and (b); and

(d) a composition comprising any one of (a), (b) and (c).

For particularly purposes, said composition may comprise at least one therapeutic agent is any one of insulin, N-acetyl cysteine (NAC), thiamine (vitamin B1), a benzodiazepine or any combination thereof and a tissue derived preparation or compound.

As discussed above, the invention provides different methods of treating, ameliorating preventing or delaying the onset of hepatic or any immune-related disorders in a subject in need. As used herein in the specification and in the claims section below, the term “treat” or “treating” and their derivatives includes substantially inhibiting, slowing or reversing the progression of a condition, substantially ameliorating symptoms of a condition or substantially preventing the appearance of symptoms of a condition, said condition is any one of an immune-related disorder and a hepatic disorder in a subject in need thereof.

The term “prevent” and all variations of this term is intended to mean the countering in advance of pathologic symptoms or a pathologic process progress. In this case it is understood that the composition is applied prior to the observation of clinical symptoms.

The terms “ameliorate” and “amelioration” relate to the improvement in the treated subject condition brought about by the compositions and methods according to the invention, wherein said improvement may be manifested in the forms of inhibition of pathologic processes associated with any one of an immune-related disorder and a hepatic disorder, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of an immune-related disorder or a hepatic disorder and their symptoms slowing their progress, further exacerbation or development, so as to appear later than in the absence of the treatment according to the invention.

By “subject in need” or “patient” it is meant any mammal who may be affected by the above-mentioned conditions, and to whom the treatment and diagnosis methods herein described is desired, including human, bovine, equine, canine, murine and feline subjects. Preferably, the patient is a human. Administering of the composition according to the method of the invention to the patient includes both self-administration and administration to the patient by another person.

The invention further encompasses the use of the composition and methods of the invention for treating any condition related to the conditions described above. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology described herein.

In a further aspect, the invention provides a pharmaceutical composition for use in a method for treating liver damage in a subject in need thereof. More specifically, such composition may comprise as an active ingredient a therapeutically effective amount of a polyethoxylated castor oil or any derivative or a combination thereof, and optionally further comprising a pharmaceutically acceptable carrier.

Still further, the invention provides a soft or an alcoholic beverage comprising at least one polyethoxylated castor oil or any derivative and at least one a natural or synthetic SE.

The invention further provides a combined composition comprising as an active ingredient at least one natural or synthetic SE and at least one polyethoxylated castor oil or any soy derivative.

It should be appreciated that the invention further encompasses the use of the combined compositions of the invention in healthy people for prevention of liver steatosis or liver disease when exposed to conditions that possibly can induce any type of liver disease.

It should be further noted that the invention provides methods and compositions for prevention of diabetes in patients with pre diabetes.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Animals

Male C57Bl/6 mice (11-12 weeks old) were obtained from Harlan Laboratories (Jerusalem, Israel) and maintained in the Animal Core of the Hadassah-Hebrew University Medical School. All mice were administered standard laboratory chow and water ad libitum and kept in a 12-hour light/dark cycle. SSB (orange flavored soda) including additives M1, OS and/or C:E were orally administered 400 μl per mouse by gavage. The experiments were performed according to guidelines of the Hebrew University-Hadassah Institutional Committee for Care and Use of Laboratory Animals and after the Committee's approval.

### Soybean Extracts

Soy extracts containing the polar (M1) and/or non-polar (OS) fractions were obtained by standard processing procedures for extracting soy oil and soy protein. M1 and OS fractions were subjected to qualitative LC-MS and ¹H-, ³¹P-NMR analyses to identify characteristic chemical profiles. Specific procedures pertaining to these methods are detailed below.

Two soy extracts were received from Solbar Israel (CHS):
OS— fraction, derived from the solvent extraction of soybeans into oil, and contains tri- and di-glycerides, free fatty acids and phosphatides;
M1—fraction which is derived from aqueous-ethanol extraction left after the solvent extraction, and contains isoflavones, sugars (oligo-, di-, mono-), and lipids (including —phosphatides, phytosterols, saponins).

The M1 (polar) fraction was obtained by standard hydro-alcoholic extraction of defatted soy milk to food soy protein. Qualitative LC-MS analysis used M1 dissolved in DMSO that was analyzed using C-18 reversed column and polar mobile phase consisting of water (modified with ammonium formate) and methanol. Qualitative ¹H-NMR analysis was carried out using different solvents to identify various constituents. M1 contained typical ratio of phosphatidylcholine (PC) and in phosphatidylinositol (PI), in declining order. More accurate ³¹P-NMR analysis showed that M1 was characterized with highly heterogeneous content of phospholipids and phosphatides. M1 was predominantly enriched in phosphatidylcholine (PC) and phosphatidylinositol (PI).

The OS (non-polar) fraction was dissolved in chloroform. The LC/MS analysis was carried out using a reversed column C-18 and non polar mobile phase consisting of methanol and ethyl acetate. The LC/MS and NMR analyses showed mainly glycerides and phospholipids, in declining order. More accurate quantitative ³¹P-NMR spectroscopy showed that OS was mainly enriched in phosphatidic acid (PA), phosphatidylethanolamine (PE) and phosphatidylcholine (PC). OS and M1 fractions were distinct by ratios of various phosphatides.

M1 and/or CE Supplementation to SSBs

MI solution consisted of 25 μg MI fraction diluted in DDW or C:E to concentration of 60 μg/ml. C:E solution was prepared from Cremophor EL (Sigma, Rehovot, Israel) in ethanol (1:1) diluted to 30% (vol./vol.) in DDW. MI supplementation to SSB was 3 μg M1 or OS/mouse.

### Concanavalin A

Concanavalin A solution (Con A; purchased from MP Biomedicals, USA) consisted of 2 mg Con A in 1 ml distilled water. Mice were intravenously (IV) injected with 250 μl Con A solution (0.5 mg/mouse).

### ALT Levels as a Parameter of Liver Damage

Mice were tested for serum Alanine transaminase (ALT) 15 hours after Con A administration. Serum ALT levels were measured by an automatic analyzer.

### GTT (Glucose Tolerance Test)

Mice undergo a glucose tolerance test on day 60. Glucose is administered orally (1 g per kg). Serum glucose measurements are performed on tail-vein blood every fifteen minutes for three hours. Glucose levels are measured by a standard glucometer.

### Statistical Analyses

Glucose serum concentration was calculated as Area Under the Curve (AUC) values at discrete time points (0, 15, 30, 60 and 180 min) after M1 and/or OS in DDW or CE administration. AUC was used as an estimate of a total glucose exposure over time under various experimental conditions, i.e. M1 and/or OS in DDW or CE administration.

Comparison of two independent groups was performed using the Student's t test. The association between two variables was assessed by calculating the Pearson and the Spearman correlation coefficients. All tests applied were two-tailed, and a p value of 0.05 or less was considered statistically significant.

### Example 1

### Beneficial and Long Term Effects of M1 Supplementation to SSB on Lowering Blood Sugar Levels

Effects of supplementation of soy-derived polar (M1) fraction on lowering blood sugar/glucose levels resulting from SSB consumption were demonstrated in a series of experiments.

### Experimental Design

The experiments included 4 groups of male C57BL/6 mice 11-12 weeks old (N=7 in each group). Table 1 shows regimens of M1 and/or C:E supplementation to SSB in various experimental groups (A-D). Total M1 supplementation was 6 μg/mouse. Glucose levels were tested at time points 0, 15, 30, 60, 90, 120 and 180 min after SSB±M1 and/or C:E consumption.

TABLE 1

| | | SSB | |
|---|---|---|---|
| Group | Number | Supplementation | Preparation |
| A | N = 7 | SSB | 3500 μl SSB + 500 μl DDW |
| B | N = 7 | SSB + M1 in C:E | 3500 μl SSB + 500 μl M1 |
| | | | in 30% C:E (240 μg/ml) |
| C | N = 7 | SSB + M1 in DDW | 3500 μl SSB + 500 μl M1 |
| | | | in DDW (240 μg/ml) |
| D | N = 7 | SSB + C:E | 3500 μl SSB + 500 μl |
| | | | 30% C:E |

Beneficial effects of M1 on lowering blood glucose levels resulting from SSB consumption are evident from FIG. 1 demonstrating that mice consuming SSB supplemented with M1, particularly in combination with C:E, had significantly lower blood glucose up to 60 min after consumption. FIG. 2 showing long term measurements of blood glucose further demonstrate that beneficial effects of M1 and C:E, alone or in combination, persist 2 to 3 hours after SSB consumption.

### Example 2

Beneficial Effects of M1 and/or OS Supplementation to SSB on Long Term Control of Blood Sugar Levels and Glucose Tolerance

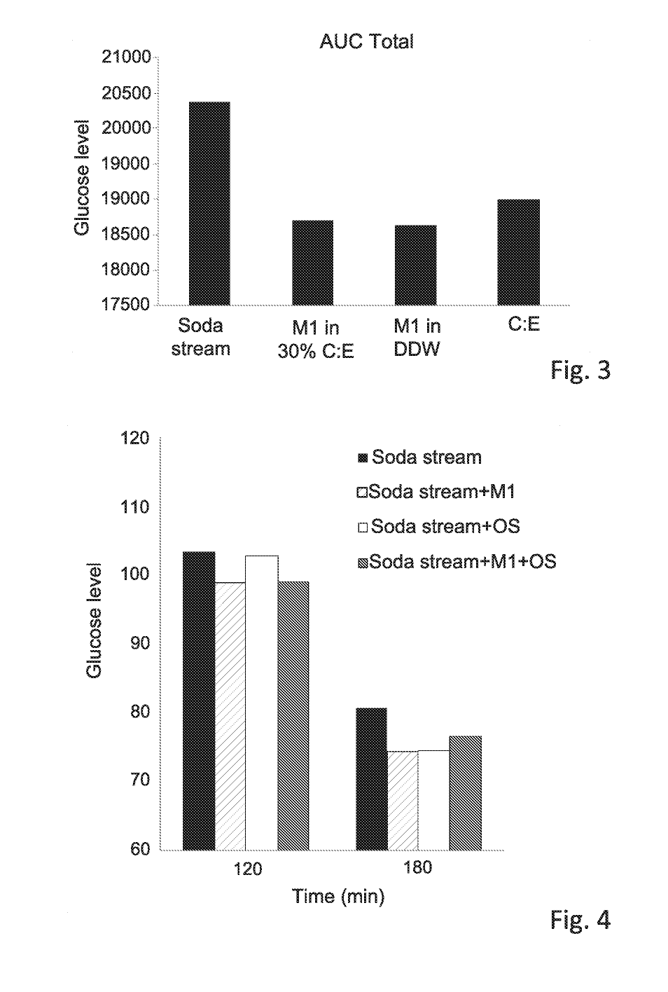

Effects of supplementation of soy-derived polar (M1) on glucose tolerance are further evident from FIG. 3 showing total AUC 120 min values. It can be seen that mice consuming SSB supplemented with M1 alone or in combination with C:E had significantly lower total glucose exposure time, which is indicative of improved glucose tolerance.

Further, effects of various combinations of soy derived polar (M1) and non-polar (OS) fractions and C:E on long term control of blood sugar levels and glucose tolerance were demonstrated in another series of experiments.

### Experimental Design

Table 2 shows regimens of M1 and/or OS supplementation to SSB in four experimental groups (A-D). Total M1 and/or OS supplementation was 6 μg/mouse. Glucose levels were tested 120 and 180 min after SSB±M1 and/or OS consumption.

Table 3 shows regimens of M1, OS and/or C:E supplementation to SSB in experimental groups (A-D). Total M1 and/or OS supplementation was 6 μg/mouse. Glucose levels were tested at 0, 15, 30, 60, 90, 120 and 180 min time points after consumption.

TABLE 2

| | | SSB | |
|---|---|---|---|
| Group | Number | supplementation | Preparation |
| A | N = 6 | SSB | 350 μl SSB + 500 μl DDW |
| B | N = 6 | SSB + M1 | 3500 μl SSB + 500 μl M1 in DDW |
| | | | (240 μg/ml) |
| C | N = 6 | SSB + OS | 3500 μl SSB + 500 μl OS in DDW |
| | | | (240 μg/ml) |
| D | N = 6 | SSB + M1 + OS | 3500 μl SSB + 250 μl M1 in DDW |
| | | | 250 μl OS in DDW (each 240 μg/ml) |

TABLE 3

| | | SSB | |
|---|---|---|---|
| Group | Number | supplementation | Preparation |
| A | N = 6 | SSB | 3500 μl SSB + 500 μl DDW |
| B | N = 6 | SSB + M1 in C:E | 3500 μl SSB + 500 μl M1 in 30% C:E |
| | | | (240 μg/ml) |
| C | N = 6 | SSB + OS in C:E | 3500 μl SSB + 500 μl OS in 30% C:E |
| | | | (240 μg/ml) |
| D | N = 6 | SSB + M1 + OS | 3500 μl SSB + 250 μl M1 in 30% C:E |
| | | in C:E | 250 μl OS in 30% C:E |
| | | | (each 240 μg/ml) |
| E | N = 6 | SSB + C:E | 3500 μl SSB + 500 μl 30% C:E |

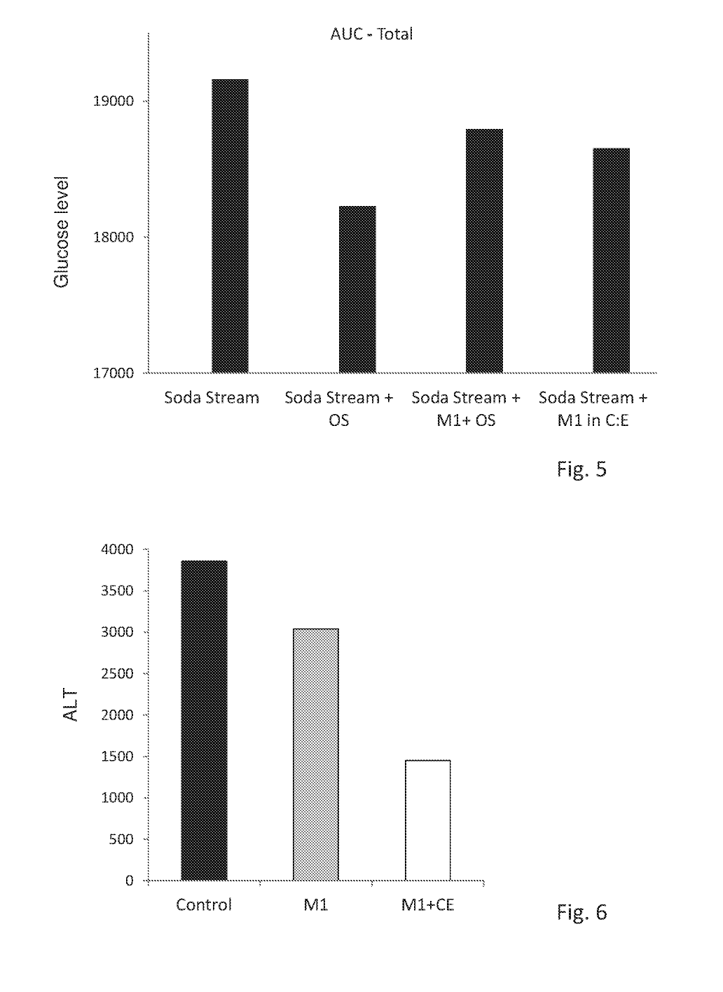

FIG. 4 showing total AUC at 120 and 180 min values of groups receiving M1 and/or OS in DDW (as described in Table 2) supplementation to SSB clearly demonstrates that both, M1 and OS, alone or in combination have beneficial effects on long term control and glucose tolerance. FIG. 5 further demonstrated that these effects may be augmented with additional supplementation of C:E. (as described in Table 3).

### Example 3

### Protective Effects of M1 and C:E on Immune-Mediated Hepatitis

Protective effects of M1 on liver function were demonstrated in an animal model of immune-mediated hepatitis induced by Con A.

### Experimental Design

The experiments included 3 groups of male C57BL/6 11-12 weeks old mice (N=5 in each group). Mice were given various treatments, M1 dissolved in DDW or 30% C:E (3 μg/mouse) 3 days prior to administering Con A (250 μl/mouse. Serum ALT levels were tested 15 hours after Con A administration. Table 4 shows various experimental groups.

TABLE 4

| Group | Number | Treatment | Con A |
|---|---|---|---|
| A | N = 5 | 30 μl DDW | + |
| B | N = 5 | 3 μg M1 in 30 μl DDW | + |
| C | N = 5 | 3 μg M1 in 30 μl 30% C:E | + |

Protective effects of M1 against immune-mediated liver damage are evident from FIG. 6 showing that mice pre-treated with M1 alone or in combination with C:E had significantly lower serum ALT levels, i.e. were more resistant to Con A insult, compared to control mice. FIG. 6 further demonstrates that M1 and C:E have an additive effect on lowering liver damage.

### Example 4

Hepatoprotective Effect of OS and M1 with CE in on the Alcohol-Induced Liver Damage

The inventors further characterized the hepatoprotective effect of the OS and M1 on liver damage using the alcohol induced liver damage mouse model. Table 5 summarizes the relevant experimental groups, including group A of naïve mice; group B receiving alcohol 6 gr/kg-300 μl of 70% Ethanol per mouse/gavage i.e. 3500 μl EtOH Abs.+1500 μl sterile water (Ethanol is equal to 6 gr/kg for 27.5 gr mouse body weight); Group C receiving 6 microgram of OS and M1 with alcohol. Mice were sacrificed after 16 hours and the levels of liver enzymes was evaluated.

TABLE 5
The effect of OS and M1 + C:E on the alcohol
induced liver damage in a mouse model,
Experimental groups

| Group | Treatment |
|---|---|
| A | naïve mice |
| N = 4 | |
| B | EtOH |
| N = 4 | |
| C | EtOH + OS M1 |
| N = 4 | 6 microgram |

AST levels measured at 16 hours in mice receiving orally ethanol (EtOH) or EtOH supplemented with OS and M1 are presented in FIG. 7. The figure clearly show a significant beneficial effect of oral co-administration of OS and M1 and alcohol in alleviating the alcohol-induced liver damage, suggesting that in these conditions OS and M1 act as liver protectors.

The protective effect of the OS and M1 combination of the invention on alcohol induced liver damage was further demonstrated on body weigh as shown in FIG. 8.

The results show body weight in grams at 16 hours in mice receiving orally ethanol (EtOH) or EtOH supplemented with OS and M1, indicating a significant beneficial effect of oral co-administration of OS and M1 and alcohol in alleviating the alcohol-induced reduction of body weight.

Still further, the inventors next examined the protective effect of the OS-M1 combination of the invention on alcohol-mediated alteration of regulatory T cells. FIG. 9 shows percentages of NKT cells (CD3+NK1.1+) and CD4+CD25+Foxp3+ regulatory T cells at 16 hours in mice receiving orally ethanol (EtOH) or EtOH supplemented with OS M1. The results show a significant beneficial effect of oral co-administration of OS and M1 and alcohol in reducing NKT cells which mediate the liver damage, and in correcting the redistribution of CD4+CD25+Foxp3+, suggesting that in these conditions OS and M1 act as immune balancers.

1.-21. (canceled)

22. A method for controlling blood sugar levels in a subject, treating liver damage, restoring liver function, treating an immune related disorder, and for treating, preventing, ameliorating, reducing or delaying the onset of acute or chronic toxic effect of a drug on an organ or tissue, said method comprises providing to a subject at least one of:
(a) at least one soy extract or any fraction thereof;
(b) at least one of at least one polyethoxylated castor oil or any derivative thereof and at least one adjuvant selected from polyethylene glycol and beta cyclo dextrin or any derivative thereof;
(c) any combination of (a) and (b); and
(d) a composition comprising any one of (a), (b) or (c).

23. (canceled)

24. The method according to claim 22, wherein said soy extract or any fraction thereof is a soy derived polar fraction, wherein said soy derived polar fraction comprises at least one of phospholipids, phosphatides or a combination thereof and wherein said phosphatides are any one of phosphatidylcholine (PC), phosphatidylinositol (PI) or a combination thereof, said polar fraction is designated M1.

25. (canceled)

26. The method according to claim 22, wherein said soy extract or any fraction thereof is a soy derived non-polar fraction, wherein said soy derived non-polar fraction comprises at least one of glycerides, phospholipids and phosphatides, and wherein said at least one of glycerides, phospholipids and phosphatides are any one of phosphatidic acid (PA), phosphatidylethanolamine (PE) and phosphatidylcholine (PC), said non-polar fraction is designated OS.

27. (canceled)

28. The method according to claim 22, wherein said derivative of polyethoxylated castor oil is Cremophore EL (C:E).

29. The method according to claim 22, wherein said (a), (b), (c) or (d) are provided in a formulation adapted for add-on to a solid, semi-solid or liquid food, beverage, food additive, food supplement, medical food, botanical drug, drug and/or a pharmaceutical compound.

30. The method according to claim 29, wherein said food and/or beverage comprises an increased content of sugar and/or alcohol or are associated with increase in blood sugar and/or alcohol level.

31. The method according to claim 22 for controlling blood sugar levels in a subject, wherein said control is inhibiting increase or decrease in blood sugar levels, improving glucose tolerance or altering insulin resistance state.

32. The method according to claim 31, for the prevention or alleviation of symptoms related to a condition associated with increased or decreased blood sugar levels, wherein said condition is any one of diabetes, obesity, hepatic disorder, pancreatic dysfunction, weight gain, alcohol intoxication, alcohol withdrawal and vertigo, any condition associated with alteration of pancreatic or liver function or tissue or organ damage.

33. The method according to claim 31, for treating a subject suffering from a disorder associated with increased or decreased blood sugar levels.

34. The method according to claim 31, for treating a subject suffering from a disorder associated with alcohol consumption.

35. The method according to claim 22 for treating, preventing, ameliorating, reducing or delaying the onset of an immune-related disorder, said method comprising the step of administering a therapeutically effective amount of at least one soy derived polar fraction and at least one polyethoxylated castor oil or any derivative thereof, any combination thereof or any composition comprising the same, wherein said immune-related disorder is any one of an inflammatory disorder, an autoimmune disorder, an infectious disease and a proliferative disorder.

36. (canceled)

37. The method according to claim 35, wherein said method further comprises the concurrent or parallel administration of at least one additional therapeutic agent, wherein said additional therapeutic agent is any one of insulin, N-acetyl cysteine (NAC), thiamine (vitamin B1), a benzodiazepine or any combination thereof and a tissue derived preparation or compound.

38. (canceled)

39. The method according to claim 22 for treating liver damage and/or restoring liver function in a subject in need thereof, said method comprising the step of administering a therapeutically effective amount of at least one soy extract or any fraction thereof and at least one polyethoxylated castor oil or any derivative thereof, any combination thereof or any composition comprising the same.

40. The method according to claim 39, wherein said subject is suffering from a liver disease, said liver disease is any one of alcoholic or autoimmune hepatitis, alcoholic or autoimmune cirrhosis, alcoholic fatty liver disease, non-alcoholic fatty liver disease (NAFLD), liver steatosis, alcoholic or nonalcoholic steatohepatits (NASH), hepatocellular carcinoma, viral, bacterial, fungal or parasitic liver disease, drug-induced liver disease and pediatric liver disease and metabolic liver disease.

41. The method according to claim 22 for treating, preventing, ameliorating, reducing or delaying the onset of acute or chronic toxic effect of alcohol consumption or of a drug and for restoring liver function.

42. (canceled)

43. A soft or an alcoholic beverage comprising a soy extract or any fraction thereof and optionally further comprising a polyethoxylated castor oil or any derivative or a combination thereof.

44. (canceled)

45. The soft or alcoholic beverage according to claim 43 wherein said soy extract or any fraction thereof is a soy derived polar fraction, said soy derived polar fraction comprises at least one of phospholipids, phosphatides or a combination thereof and wherein said phosphatides are any one of phosphatidylcholine (PC), phosphatidylinositol (PI) or a combination thereof, said polar fraction is designated M1.

46. The soft or alcoholic beverage according to claim 43 wherein said soy extract or any fraction thereof is a non-polar fraction, said fraction is a non-polar fraction comprising at least one of glycerides, phospholipids and phosphatides, said non-polar fraction is designated OS.

47. A combined composition comprising as an active ingredient at least one soy derived polar fraction and at least one polyethoxylated castor oil or any derivative thereof.

48.-49. (canceled)

50. The method according to claim 22 for enhancing and augmenting the therapeutic effect of at least one therapeutic agent in a subject treated with said at least one therapeutic agent, the method comprises providing to a subject a therapeutically effective amount of at least one of:
(a) at least one SE or any fraction thereof;
(b) at least one polyethoxylated castor oil or any derivative thereof and/or optionally, at least one adjuvant selected from polyethylene glycol and beta cyclo dextrin or any derivative thereof;
(c) any combination of (a) and (b); and
a composition comprising any one of (a), (b) or (c).

51.-55. (canceled)
